(19) United States
(12) Patent Application Publication
(10) Pub. No.: US 2004/0006777 A1
(43) Pub. Date: Jan. 8, 2004

(54) Human lymphoid protein tyrosine phosphatases

(75) Inventor: Chaim M. Roifman, North York (CA)

Correspondence Address: ROPES & GRAY LLP, ONE INTERNATIONAL PLACE, BOSTON, MA, 02110-2624, US

(73) Assignee: HSC Research and Development Limited Partnership, Toronto (CA)

(21) Appl. No.: 10/309,423

(22) Filed: Dec. 3, 2002

Related U.S. Application Data
(63) Continuation of application No. 09/600,358, filed on Sep. 25, 2000, now abandoned.
Application No. 09/600,358 is a 371 of international application No. PCT/CA99/00038, filed on Jan. 18, 1999.

(30) Foreign Application Priority Data
Jan. 16, 1998 (CA) 2,220,853

(51) Int. Cl. C12Q 1/68; A01K 67/00; C07H 21/04; C12N 9/16; C12P 21/02; C12N 5/06

(52) U.S. Cl. 800/8; 435/6; 435/196; 435/320.1; 435/325; 536/23.2; 435/69.1

(57) ABSTRACT

The invention provides a human gene encoding two new cytoplasmic phosphatases, Lyp1 and Lyp2, as well as the proteins and methods for preparing and using the described gene and proteins.

### FIELD OF THE INVENTION

This invention relates to a human phosphatase gene (Lyp) which encodes an intracellular tyrosine phosphatase (Lyp1) and an isoform of Lyp1, called Lyp2. More particularly, it relates to the cDNA sequence of human Lyp1 and Lyp2, the protein products and the expression, role and use of these phosphatases in humans.

### BACKGROUND OF THE INVENTION

Protein tyrosine phosphorylation, a key mechanism of cellular signal transduction, is regulated by the action of both protein tyrosine kinases (PTKs) and protein tyrosine phosphatases (PTPases). Originally PTKs were believed to control the process of tyrosine phosphorylation, with a small number of PTPases playing largely housekeeping roles. Unexpectedly, the structural diversity of the growing number of PTPases has called this idea into question and it has become apparent that PTPases have important roles in the regulation of growth and differentiation in both normal and neoplastic cells (1,2).

All PTPases contain a catalytic domain of approximately 200-300 residues including a subset of highly conserved amino acids that play a role in substrate recognition and tyrosine dephosphorylation (3). The PTPases family can be divided broadly into two major classes: membrane bound, receptor-like or receptor phosphatases and non-receptor or intracellular phosphatases (4, 5). Both types can be further subdivided into subfamilies based on their sequence similarities and non-catalytic domain structure motifs (6,7).

The receptor PTPases have one or two intracellular phosphatase domains and often have Ig-like domains and fibronectin-like extracellular regions (6) that play a role in cell-cell or cell-matrix interactions (8). In fact, some receptor PTPases appear to participate in homophilic and heterophilic binding interactions suggestive of a role in cell guidance and contact inhibition (7,8).

The non-receptor phosphatases display various intracellular localizations determined by amino acid sequences outside the catalytic domain (9, 35, 40). Some contain conserved non-catalytic domains such as the Src homology 2 (SH2) and SH3 domains allowing them to interact with a variety of tyrosine phosphorylated proteins and proteins containing proline rich sequences (19, 10, 11). Cytoplasmic PTPases have been found associated with a variety of PTKs including CSK and the Jak kinases, and a number of cytokine and antigen receptors (7, 11, 31).

Several lines of evidence indicate that within the immune system, PTPases are essential for lymphocyte development and activation. CD45, a transmembrane phosphatase expressed exclusively in hematopoietic cells (12), is required for antigenic activation of B and T lymphocytes (13, 14). In addition, evidence from CD45-deficient mice indicates that CD45 also plays a pivotal role in thymic development and T cell apoptotic response to T cell receptor engagement (15, 16). Recent studies have suggested that the hematopoietic-specific intracellular phosphatase-SHP1 (SH2 containing PTPase) negatively regulates signaling through association with the B cell receptor, PcγRIIB1 (17) and the IL-3 receptor 3 chain (18). SHP1 also participates in T cell signalling events through dephosphorylation of the T cell receptor (TCR), p56^{lck} and ZAP-70 (19). Mutations in the murine motheaten locus coding the SHP1 protein result in severe combined immunodeficiency and systemic autoimmunity, as well as many other hematopoietic abnormalities (20). Furthermore, expression of HePTP, a cytoplasmic hematopoietic-specitic PTPase, is induced in lymphocytes stimulated by phytohemagglutinin, concavalin A, lipopolysaccharide and anti-CD3 (21), suggestive of a role in lymphocyte activation pathways.

### SUMMARY OF THE INVENTION

A human gene encoding a novel cytoplasmic phosphatase protein, Lyp1, has been identified and designated Lymphoid Protein Tyrosine Phosphatase gene (Lyp). The Lyp gene has been localized to human chromosome 1p13.

The phosphatase Lyp1 is involved in lymphocyte growth and development and is predominantly expressed in lymphoid cells. Lyp1 is an intracellular 105 kDa protein containing a single tyrosine phosphatase catalytic domain.

In addition, an isoform of Lyp1 has been identified and designated Lyp2. This isoform is a product of C-terminal alternative RNA splicing resulting in a smaller 85 kDa protein.

The cDNA sequences encoding Lyp1 and Lyp2 have been cloned, sequenced and expressed to provide the respective proteins. The proteins are most highly expressed in lymphoid tissues including spleen, lymph nodes, peripheral leukocytes, tonsil, bone marrow, thymocytes and in both immature and mature B and T lymphocytes. Lyp1 expression has been demonstrated to be induced by activation of resting peripheral T lymphocytes with PHA or anti-CD3. Lyp1 has also been demonstrated to bc constitutively associated with the proto-oncogene c-Cbl (a protein which is recognized to be important in the regulation of the Zap family kinases) in thymocytes and T cells. Overexpression of Lyp1 reduced Cbl tyrosine phosphorylation, suggesting it may be a substrate of the phosphatase. Lyp 1 has also been demonstrated to down-regulate the activity of the T cell tyrosine kinase Zap-70, with little effect on Syk kinase. Lyp1 strongly down-regulates Lyn kinase activity, while Fyn function is unaffected. In B cells, Lyp is constitutively bound to the Syk kinase and inducibly binds a number of phosphorylated proteins after stimulation of the cell through the B cell receptor.

It therefore appears that the protein products of the Lyp gene are important for regulation of T cell antigen receptor signalling and cytokine receptor signalling.

In accordance with one embodiment, the invention provides an isolated polynucleotide comprising a nucleotide sequence encoding a Lyp protein.

In accordance with a further embodiment, the invention provides an isolated polynucleotide which encodes a Lyp protein having an amino acid sequence of greater than 70% overall identity to the amino acid sequence of Table 2.

In accordance with a further embodiment, the invention provides an isolated polynucleotide which encodes a Lyp protein having an amino acid sequence of greater than 70% overall identity to the amino acid sequence of Table 4.

In accordance with a further embodiment, the invention provides an isolated polynucleotide which encodes a Lyp protein having an amino acid sequence with at least 80% overall identity, preferably at least 90% overall identity to the amino acid sequence of Table 2.

In accordance with a further embodiment, the invention provides an isolated polynucleotide which encodes a Lyp protein having an amino acid sequence with at least 80% overall identity, preferably at least 90% overall identity to the amino acid sequence of Table 4.

In accordance with a further embodiment, the invention provides a nucleotide sequence comprising at least 10, preferably 15 and more preferably 20 consecutive nucleotides of Sequence ID NO:1 or Sequence ID NO:3.

In accordance with a further embodiment, the invention provides a substantially purified Lyp protein.

In accordance with a further embodiment, the invention provides a substantially purified protein having an amino acid sequence of greater than 70% overall identity to the amino acid sequence of Table 2.

In accordance with a further embodiment, the invention provides a substantially purified protein having an amino acid sequence of greater than 70% overall identity to the amino acid sequence of Table 4.

In accordance with a further embodiment, the invention provides a substantially purified protein having an amino acid sequence with at least 80% overall identity, preferably at least 90% overall identity, to the amino acid sequence of Table 2.

In accordance with a further embodiment, the invention provides a substantially purified protein having an amino acid sequence with at least 80% overall identity, preferably at least 90% overall identity, to the amino acid sequence of Table 4.

In accordance with a further embodiment, the invention provides a peptide comprising at least 5, preferably 10, more preferably 20 consecutive amino acids of Sequence ID NO:2 or Sequence ID NO:4.

In accordance with a further embodiment, the invention provides a peptide comprising at least one functional domain of a Lyp protein.

In accordance with a further embodiment, the invention provides a peptide comprising at least one antigenic determinant of a Lyp protein.

In accordance with a further embodiment, the invention provides an antibody which binds specifically to a Lyp protein.

In accordance with a further embodiment, the invention provides a method for screening a candidate compound for an ability to increase or decrease the phosphatase activity of a Lyp protein comprising

(a) providing an assay system for assaying Lyp phosphatase activity;

(b) assaying Lyp phosphatase activity in the presence or absence of the candidate compound; and

(c) determining whether the Lyp phosphatase activity was higher or lower in the presence of the candidate compound than in its absence.

In accordance with a further embodiment, the invention provides a method for screening a candidate compound for ability to modulate expression of a Lyp gene comprising

contacting a cell with a candidate compound, wherein the cell includes a regulatory region of a Lyp gene operably joined to a coding region; and

detecting a change in expression of the coding region.

In accordance with a further embodiment, the invention provides a non-human animal wherein a genome of said animal, or of an ancestor thereof, has been modified by a modification selected from the group consisting of:

(a) knockout of a Lyp gene; and

(b) insertion of a polynucleotide encoding a heterologous Lyp gene.

In accordance with a further embodiment, the invention provides a pharmaceutical composition comprising an active ingredient selected from the group consisting of:

(a) an isolated nucleotide sequence encoding a Lyp protein;

(b) a substantially purified Lyp protein;

(c) a substantially purified antibody which binds specifically to a Lyp protein and a pharmaceutically acceptable carrier.

In accordance with a further embodiment, the invention provides a method for treating a subject having a deficiency of Lyp activity comprising administering to the subject an effective amount of an agent selected from the group consisting of:

(a) an isolated nucleotide sequence encoding a Lyp protein;

(b) a substantially purified Lyp protein.

In accordance with a further embodiment of the invention, a method is provided for identifying allelic variants or heterospecific homologues of a Lyp1 gene comprising

(a) choosing a nucleic acid probe or primer capable of hybridizing to a human Lyp1 gene sequence under stringent hybridization conditions;

(b) mixing said probe or primer with a sample of nucleic acids which may contain a nucleic acid corresponding to the variant or homologue;

(c) detecting hybridization of the probe or primer to the nucleic acid corresponding to the variant of homologue.

In accordance with a further embodiment of the invention, a pharmaceutical composition is provided comprising an active ingredient selected from the group consisting of:

an antisense sequence which hybridizes to a human Lyp1 nucleotide sequence or to a transcript of the sequence;

a substantially pure antibody which binds selectively to human Lyp1 or Lyp2 protein and a pharmaceutically acceptable carrier;

a mimetic of human Lyp1 or Lyp2 protein;

a functional analog of human Lyp1 or Lyp2 protein;

an inhibitor of human Lyp1 or Lyp2 protein activity; and

an agent capable of altering the phosphorylation state of human Lyp1 or Lyp2 protein.

In accordance with a further embodiment of the invention, a method is provided of screening for an agent useful in treating a disorder characterized by an abnormality in a phosphorylation signaling pathway of lymphoid cells, wherein the pathway involves an interaction between a human Lyp1 or Lyp2 protein and a human Lyp1 or Lyp2 activator, comprising screening potential agents for ability to disrupt or promote the interaction as an indication of a useful agent.

In accordance with a further aspect of the invention, a method is provided of preventing or treating a disorder in a mammal characterized by an abnormality in an intracellular phosphorylation signaling pathway of lymphoid cells, wherein the pathway involves an interaction between a human Lyp1 or Lyp2 protein and a human Lyp1 or Lyp2 substrate, comprising the step of disrupting or promoting said interaction in vivo.

### BRIEF DESCRIPTION OF THE DRAWINGS

Certain embodiments of the invention are described, reference being made to the accompanying drawings, wherein:

FIG. 1 shows a schematic diagram of Lyp1 and Lyp2 deduced from the cDNA clones. Boxes indicate the open reading frame, with thin lines representing the 5′ and 3′ untranslated regions. The six overlapping cDNA clones (bold black lines) obtained from a human thymus cDNA library are shown under the schematic structures of the cDNAs.

FIG. 2 shows an alignment of Lyp1 and Z70PEP (Sequence ID NO:5) amino acid sequences. The PTPase domain is indicated by brackets. An arrow indicates the end of the amino acid sequence shared by Lyp1 and Lyp2 and the beginning of the unique C-terminal sequence of Lyp1. The NXXY motif is indicated by line above the sequence. The four potential SH3 domain binding sites are also indicated (asterisks). A consensus sequence is shown below the alignment. The unique seven amino acids of Lyp2 are shown in the box below the alignment.

FIG. 3A (left panel) demonstrates the regional mapping of the Lyp gene by fluorescence in situ hybridization to normal human lymphocyte chromosomes counterstained with DAPI. Biotinylated cDNA probe was detected with avidin-fluorescein isothiocyanate (FITC). Separate images of DAPI counterstained metaphase chromosomes and of Lyp cDNA probe hybridization signals were captured and overlaid electronically. Part of a representative metaphase preparation is shown to indicate the position of the Lyp probe FISH signals that are visible as two yellow fluorescent spots on the p arm of chromosome 1. FIG. 3A, right panel, shows a DAPI banded chromosome. FIG. 3B shows a schematic ideogram of the DAPI banded chromosome of FIG. 3A, right panel, indicating that the Lyp1 probe hybridizes to band 1p13.

FIG. 4 demonstrates that Lyp2 is a result of alternative splicing of the Lyp gene. A, schematic map of the PCR strategy used. Primer 1 corresponds to the last 20 nucleotides shared by both Lyp1 and Lyp2 sequences, primer 2 to Lyp2 untranslated area and primer 3 to the beginning of the novel Lyp1 sequence, immediately downstream of primer 1. (see also C). B. The results of the PCT amplification on genomic DNA. Lane 1, DNA size markers, Lane 2, a product of 3.5 kb was amplified with primers 1 and 3. Lane 3, is a product of 100 bp was amplified with primers 1 and 2. C. Schematic map of Lyp1 splicing. The sequences before the vertical line represent the splice donor site, while the nucleotide sequences after it are the Lyp1 intronic sequence which code for the unique C-terminal seven amino acids, stop codon (asterisk) and untranslated sequence (lower case letters) of Lyp1. A white box represents the common cDNA sequence shared by Lyp1 and Lyp2, the black and the light gray boxes representing the unique sequences of each cDNA (Lyp1 and Lyp2 respectively).

FIG. 5 shows the expression profile of Lyp1 and Lyp2 transcripts. The size of RNA markers are indicated in kb. A. Human tissues of various origin. B/C. Immune relevant human tissues.

FIG. 6 shows an immunoblot of transfected COS-7 cells. T7 tagged Lyp1 (A) or Lyp2 (B) were transfected into COS-7 cells and immunoprecipitated with anti-Lyp or anti-T7 antibody and blotted with anti-T7. (A): Lyp1 transfection results in a transfected protein of 105 kD and a probable degradation product of 96 kD, while (B): shows Lyp2 as a protein of 85 kD.

FIG. 7 shows in vivo translation of Lyp1 and Lyp2 proteins, COS-7 cells were transiently transfected with HA-tagged Lyp1 and Lyp2 cDNAs and protein expression analyzed by Western blotting with anti-HA tag antibodies. Lane 1, pcDNA3. Lane 2, pcDNA3 Lyp1. Lane 3, pcDNA3 Lyp2. Molecular mass markers are shown in kDa.

FIG. 8 shows the relative quantification of Lyp1 and Lyp2 transcripts in thymocytes cDNA by competitive PCR. Different concentration of competitor DNA were added to fixed amount of sample cDNA. The results of PCR amplification products of (A) 26 cycles with specific primers to Lyp1 (B) 35 cycles with specific primers to Lyp2. The internal control concentrations are indicated below the pictures in Pico-Molar.

FIG. 9 shows the localization of Lyp1 and Lyp2 in transiently transfected COS-7 cells by immunofluorescence. COS-7 cells were transiently transfected with HA-tagged Lyp1 and Lyp2 cDNAs and immunofluorescence was performed using a monoclonal antibody against HA tag. Magnification 1000×. (A) Cells transfected with HA-Lyp2 cDNA. (B) Cells transfected with HA-Lyp1 cDNA.

FIG. 10 shows Lyp protein expression in lymphoid and myeloid cell lines. Lyp was immunoprecipitated from cell lines (10⁷ cells) and blotted with Lyp antibodies. A protein band of 105 kD corresponding to Lyp1 could be detected in Jurkat, Daudi, Ramos, A1 and G2 cells, while U937 and K562 do not appear to have detectable amounts of Lyp. (PB)-Pre-immune serum control.

FIG. 11 shows the expression of Lyp proteins in resting and activated T cells. A. Lyp was precipitated from thymocytes (80×10⁶ cells), peripheral blood T cells (25×10⁶ cells) and tonsil T cells (10×10⁶ cells) and immunoblotted with anti-Lyp. Pre-immune serum controls (PB) are presented in each case. A band of 105 kD is present in each sample and a band of 85 kD can be seen only in resting peripheral T cells. B. Lyp was immunoprecipated from peripheral blood T-cells (25×10⁶ cells) before and after stimulation with anti-CD3 (2.5 μg/ml) or PHA over a period of 48 hours. There is increased 105 kD Lyp1 expression, while the 85 kD protein appears to be down regulated.

FIG. 12 shows the measurement of Lyp1 phosphatase activity. Anti-Lyp immunoprecipitates from untransfected and pcDNA3-Lyp1 transfected cells were prepared in pervanadate free lysis buffer and incubated with ³³P labelled substrate Raytide. At the indicated time points reactions were stopped by the addition of charcoal and the free ³³P released from the peptide and now present in the supernatant measured by liquid scintillation counting.

FIG. 13 demonstrates the involvement of Lyp1 in TCR signaling. (A) Lyp immunoprecipitates from thymocytes (80×10⁶ cells) stimulated with anti-CD3 were blotted with antiphsophotyrosine. A single phosphorylated band of 116 kD was detected co-immunoprecipitating with Lyp. Lyp protein loading was quantitated by anti-Lyp western blot after stripping. (B) Immunoblotting with anti-Cbl identified the 116 kD phosphorylated protein as Cbl, while immunoblotting with anti-FAK or anti-p110 (subunit of PI-3 kinase) showed them not to be associated with Lyp. (C) Lyp1 was transfected into COS-7 cells and Cbl immunoprecipitates prepared from these and untransfected cells. Western blotting was performed with Lyp antibodies. The position of Lyp is indicted by an arrow. Cbl immunoprecipitates were also prepared and blotted with anti-phosphotyrosine (D), and then anti-Cbl after stripping.

FIG. 14 shows the immunoprecipitation of the Lyp1, ZAP-70 and FYN proteins from (a) COS-7 monkey epithelial cell line or (B) the 293T human epithelial cell line, the cell lines being transfected with the cDNA for Lyp1, ZAP-70 or Fyn in the eucaryotic expression vector pcDNA3. Western blotting was performed with anti-phosphotyrosine antibodies and chemiluminescent detection reagents. In both cell lines, Lyp1 co-transfection resulted in a reduction in Zap-70 phosphorylation while Fyn was unaffected. Lyp1 down-regulated Zap-70 after activation by Fyn in 293-T cells (B), lanes 3 and 4. Syk was unaffected by Lyp1 (C).

FIG. 15 shows shows the immunoprecipitation of the Lyp1, Jak3, Syk and Fyn proteins from COS-7 monkey epithelial cell line, the cell line being transfected with the cDNA for Lyp1, Jak3, Syk, or Fyn in the eucaryotic expression vector pcDNA3. Syk was also co-transfected with Lyp-N, a catalytically inactive form of Lyp 1. Western blotting was performed with anti-phosphotyrosine antibodies and chemiluminescent detection reagents. Lyp1 reduced the tyrosine phosphorylation of Jak3 (C) while having little effect on Syk (D). No effect was seen when Syk was co-transfected with Lyp-N (D).

In the drawings, preferred embodiments of the invention are illustrated by way of example. It is to be expressly understood that the description and drawings are for the purpose of illustration and as an aid to understanding, and are not intended as a definition of the limits of the invention.

### DESCRIPTION OF THE INVENTION

A novel intracellular human phosphatase gene, Lyp, has been isolated and identified. Lyp is predominantly expressed in the lymphoid cell lineages. Lyp is a member of the PEST phosphatase family and is most closely related to the murine phosphatase Z70PEP. Hydropathy analysis has indicated that Lyp contains no obvious signal sequence or hydrophobic segments and thus apparently encodes a cytoplasmic protein containing a single catalytic phosphatase domain. The non-catalytic portions of the phosphatase contain areas of high proline, glutamic acid, serine and threonine content (PEST sequences). There also appear to bc other formally recognized functional domain structures within the remainder of the protein. A short linear amino acid sequence also found in PEP has been demonstrated to bind the murine phosphatase to the cytoplasmic tyrosine kinase csk. There are otherwise several areas of high proline content which potentially may be recognized by SH3 domains.

Two forms of messenger RNA have been isolated for Lyp. The longer encodes the entire protein of 808 amino acids, Lyp1, while the second shorter form arises from alternative splicing of the RNA and encodes Lyp2, which has 692 amino acids.

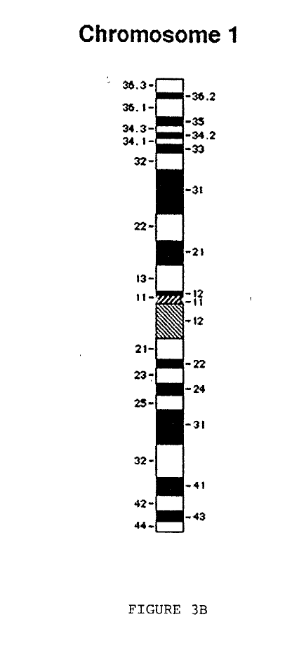

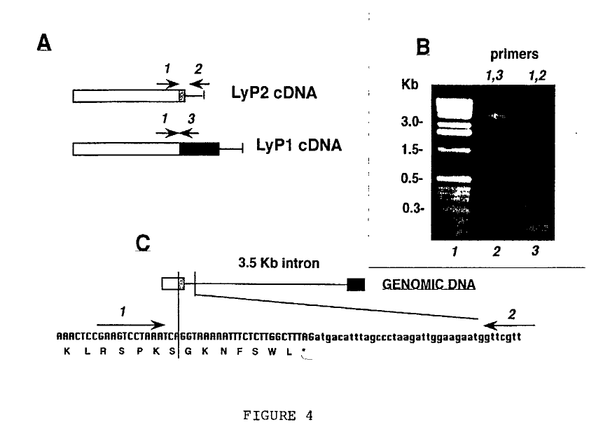

A 3.5 kb intronic sequence of Lyp1 was found to contain an alternative exon, coding for the C-terminal 7 amino acids of Lyp2, and at least part of its 3′ untranslated area (FIG. 4). The Lyp2 coding sequence consequently reads into this intronic sequence until a termination codon is encountered. As a result, the two proteins have the first 685 amino acids in common, and the catalytic domain of the two forms is identical, as is most of the non-catalytic area. However, the final 123 amino acids of Lyp1 are absent in Lyp2 and are replaced by seven unique residues. This is highly suggestive of major differences in the regulation of the activity of the two isoforms. Studies of expression of the proteins suggests that Lyp2 may only be present in resting lymphoid cells, while Lyp1 expression is increased upon activation.

Two novel intracellular protein tyrosine phosphatase cDNA sequences have been isolated from a human thymus cDNA library; the first for Lyp1 (GenBank Accession No. AF 001846) and the second for its splice variant, Lyp2 (GenBank Accession No. AF001847). The cDNA sequences for Lyp1 and Lyp2 (Sequence ID Nos: 1 and 3) are shown in Tables 1 and 3, respectively. The corresponding amino acid sequences for Lyp1 and Lyp2 (Sequence ID Nos:2 and 4) are shown in Tables 2 and 4 respectively.

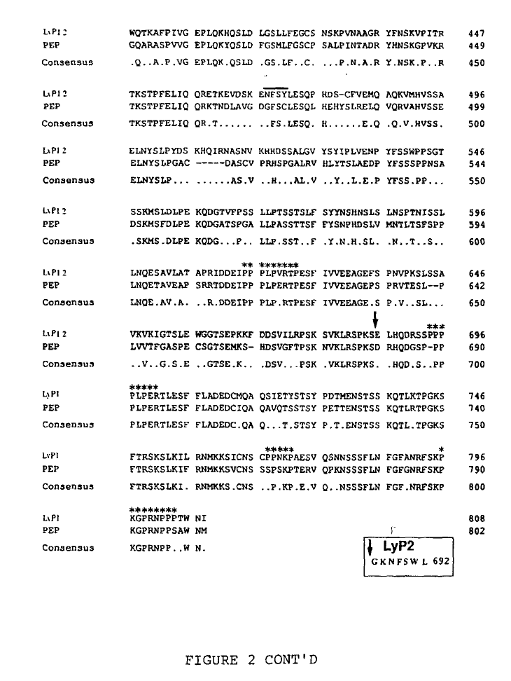

Sequence analysis of Lyp1 reveals significant homology with the murine phosphatase Z70PEP, an intracellular PTPase widely expressed in hematopoietic tissues (10). Lyp1 shares an overall amino acid sequence identity of 70% with Z70PEP (FIG. 2). While there is 89% identity between the catalytic domain of Lyp1 and Z70PEP, significantly less homology is observed within the non catalytic portion (61%), which clearly contains a large area of unique sequence. Within this low homology area, Lyp1 contains four proline rich sequences which are also present in PEP (FIG. 2), forming putative PXXP and class 11 (XPPLPXR) SH3 domain binding motifs (10, 31). It has been demonstrated that an association exists between one of the proline-rich motifs of PEP (PPPLPERTP, also present in Lyp) and the SH3 domain of the protein tyrosine kinase p50csk (32). Experiments also show Lyp1 to associate with csk in T cells (data not shown). The Lyp1 non-catalytic domain also contains a large area of unique sequence, including an NXXY motif (FIG. 2). When tyrosine phosphorylated, this motif may be recognized by a phosphotyrosine binding (PTB) domain (29) found in adaptor proteins such as IRS, Shc and cbl.

The murine Z70PEP also possesses several consensus PEST sequences (hence its name [PEST]-domain Phosphatase)(10). PEST sequences contain an unusually high percentage of proline (P), glutamic/aspartic acid (E/D), serine (S), and threonine (T) residues. An analysis of the Lyp1 sequence using the program PEST-FIND (PC analysis software; Oxford Molecular Group, Oxford) indicated the presence of only a single PEST region (amino acids 702-736), while five were confirmed in Z70PEP.

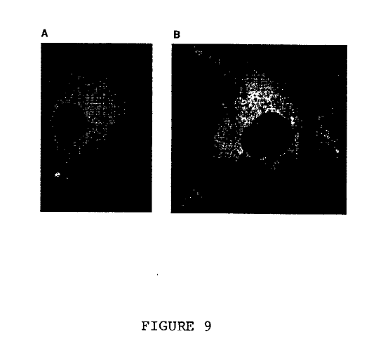

Through immunofluorescent staining of transiently transfected Cos-7 cells, it was determined that both Lyp1 and Lyp2 show a similar pattern of diffuse cytoplasmic staining (FIG. 9).

The significance of the alternative C-terminal sequences of Lyp1 and Lyp2 remains unclear, but there are several differences between the C-terminal tails that may be key in revealing functional divergence. The C-terminus of Lyp1, but not Lyp2, contains a consensus sequence XS/TPXK/R (⁷⁴¹KTPGK⁷⁴⁵) recognized by the p34^{cdc2} kinase (41), a cell cycle regulatory kinase (42), suggesting that Lyp1 may be phosphorylated in a cell cycle dependent manner. Lyp1 also contains four potential SH3 domain binding sites, compared to a single motif in Lyp2; suggesting the isoforms may interact with different sets of SH3 domains.

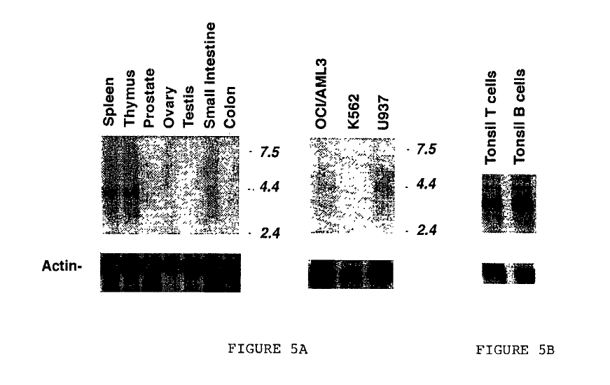

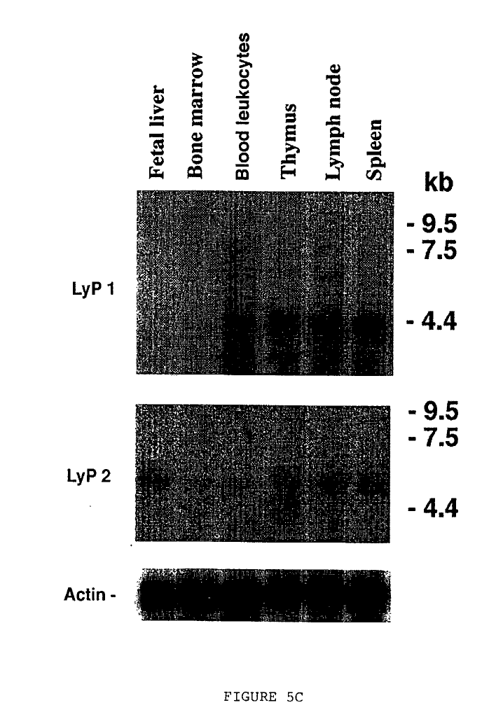

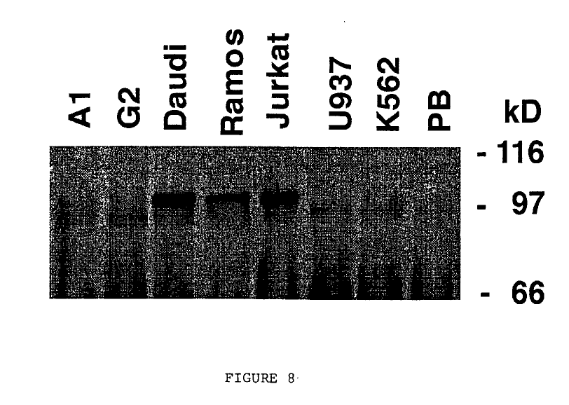

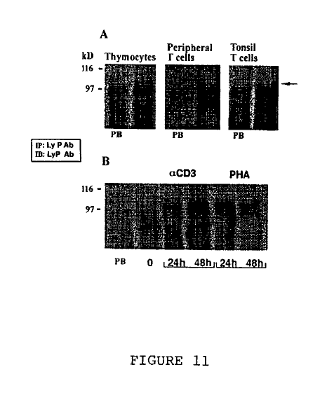

The pattern of Lyp1 expression observed by Northern blotting suggests that it is preferentially expressed in lymphoid cells (FIG. 5A,B,C), particularly in thymocytes and mature B and T cells. A low level of Lyp1 expression was also seen in tissues rich in lymphoid infiltrates, such as the small intestine and appendix. The pattern of Lyp1 protein expression detected by antibodies in human hematopoietic cell lines correlated well with Lyp1 mRNA expression (FIG. 11). This pattern of expression suggests that Lyp1 may play a role in the regulation of aspects of both early and late states of T cell differentiation. The lack of expression in fetal liver tissue, which contains a large population of pre-B cells, may suggest a different role in the biology of B cell development. The mRNA expression of Lyp1 and its isoform, Lyp2, was differentiated by the use of more specific probes. While Lyp2 was present at lower levels than Lyp1 in all lymphoid tissues examined, Northern blot analysis indicated significant expression of Lyp2 in fetal liver tissue. Lymphoid mRNAs hybridized with a probe specific for the unique C-terminal of Lyp1 revealed the same pattern of expression seen in Northern blots obtained by using a cDNA fragment common to both Lyp forms (not shown). This suggests that Lyp2 expression in lymphoid cells is extremely low, below the threshold of detection of Northern blotting. This suggestion was confirmed by semi-quantitative PCR comparison of Lyp1 and Lyp2 expression. In thymocytes, the expression of Lyp1 was found to be 100 fold greater than that of Lyp2 (FIG. 8). Similar results were obtained from other lymphoid cells (not shown).

Resting peripheral T lymphocytes demonstrated expression of an 85 kD protein recognized by the Lyp specific antibodies. Stimulation of T lymphocytes with PHA or anti-CD3 resulted in the induction of the Lyp1 protein, with a simultaneous down regulation of the 85 kD protein (FIG. 11). The 85 kD protein is believed to be Lyp2 on the basis of its apparent molecular weight and the fact that both Lyp antibodies can recognize it. This finding suggests that Lyp2 may play an important role in resting cells, since thymocytes, tonsil T cells and lymphoid cell lines, which are activated cells, do not express the protein.

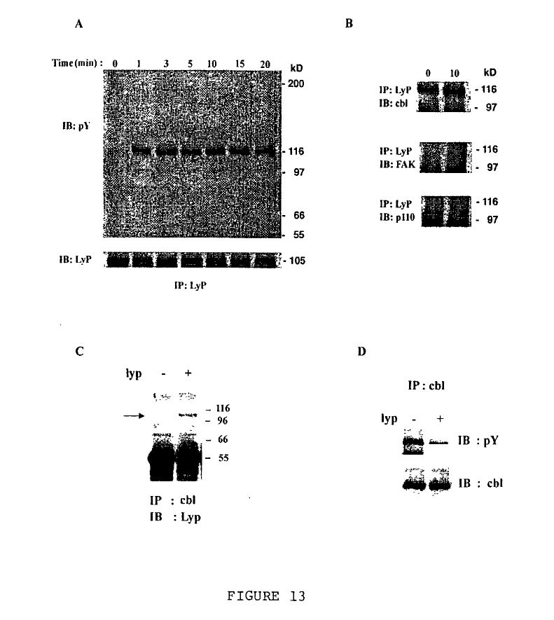

Anti-CD3 stimulation of thymocytes was found to induce the association of a 116 kD phosphorylated protein with Lyp1. Western blotting of Lyp immunoprecipitates identified the phosphorylated band to be the proto-oncogene c-Cbl. Although inducibly phosphorylated, cbl was found to be constitutively associated with Lyp1. From previous studies it is known that Cbl is heavily tyrosine phosphorylated following TCR stimulation (58) and can associate with the Syk and ZAP tyrosine kinases, negatively regulating their activities (59-63). Treatment of Jurkat cells with the phosphatase inhibitor pervanadate leads to a marked increase in the phosphorylation of Cbl (61) suggesting that tyrosine phosphatases keep Cbl in a basally dephosphorylated state. It has been now demonstrated that Lyp1 is basally associated with Cbl in thymocytes; this interaction was confirmed in Jurkat cells (data not shown) and in COS cells by transfection, where Cbl phosphorylation was also reduced by Lyp1 overexpression (FIG. 13). This strongly suggests that Lyp may play a role in regulating Cbl activity through modulation of its tyrosine phosphorylation status. As Cbl is an adaptor protein which associates with numerous protein tyrosine kinases, it is possible that Lyp may play a role in the regulation of these proteins (62). Although direct tyrosine phosphorylation of Lyp1 was not detected, a minor variant (EPNY) of the Cbl PTB domain consensus binding motif (D(N/D)XpY) is present in the non-catalytic domain, which could form the basis for interaction. Alternatively, in the absence of other identifiable interactive domains in either protein, a multiple SH3 domain adaptor protein such as Grb2 may serve to link Lyp and Cbl.

Thus it appears that Lyp1 is constitutively associated in T cells with the proto-oncogene c-Cbl, a protein which is recognized to be important in the regulation of the Zap family kinases. In B cells, Lyp is constitutively bound to the Syk kinase and inducibly binds a number of phosphorylated proteins after stimulation of the cell through the B cell receptor.

It has also been demonstrated that Lyp1 reduces phosphorylation of, and thereby reduces the activity of, the T cell tyrosine kinase, Zap-70, while it has little effect upon the closely related Syk kinase, possibly even elevating its activity. A similar selective activity is seen with members of the src family of kinases. Lyp1 strongly down-regulates Lyn kinase activity, while Fyn function is unaffected. The ability to turn off Zap-70 and Lyn strongly suggests a role for Lyp in regulating antigen receptor signalling, as these kinases are central to the signal transduction cascades. Overexpression of Lyp1 in T cells appears to interfere with activation of the Zap-70 kinase through the T cell receptor.

Lyp1 also reduced phosphorylation of, and thereby reduced the activity of, the cytoplasmic Jak3 tyrosine kinase and prevented it from phosphorylating the insulin receptor substrate proteins. Thus, Lyp is also in a position to regulate signal transduction through a number of the cytokine receptors. Activation of Jak kinases appears to be a primary event after ligand binding to cytokine receptors and absolutely necessary for signal transduction.

Co-transfection of COS cells with encoding sequences for Lyp1 and Zap70 kinase, or for Lyp1 and Jak3 kinase, led to complete dephosphorylation of Zap70 and of Jak3, suggesting that Lyp1 regulates Zap70 and Jak3 activities in T cells. Co-transfection with Lyp1 and Syk kinase, a relative of Zap70, showed no Syk dephosphorylation, indicating a selective effect of Lyp1 on Zap70 and Jak3. This selectivity was further demonstrated by experiments performed by including Lyp1 and src family kinases, such as Ick and fyn, which again showed no dephosphorylation.

T lymphocytes lacking functional Zap 70 protein do not respond satisfactorily to antigenic stimulation and since antigenic stimulation is required for normal T cell maturation, such lymphocytes fail to mature properly (Arpaia et al., (1994), Cell, v. 76, pp. 947-958; Perlmutter, R., (1994), Nature, v. 370, p. 249). The importance of Zap 70 kinase in T cell antigen receptor signalling also means that if one can interfere with or prevent Zap 70 kinase activity, one can modulate T cell activation and proliferation in situations where T cell activation and proliferation is excessive or undesired. Stimulators or activators of Lyp1 could be used as drugs which, by reducing Zap70 activity, could reduce or block T cell activation.

Similarly, over-expression of Lyp1 could be used to control T cell activation.

The proliferation of T cells also depends on IL2 receptor signal transduction, which involves Jak3 kinase activation. Hence increasing Lyp1 activity will reduce or prevent T cell proliferation by reducing Jak3 kinase activity.

Over-expression of, or stimulation of the activity of, Lyp1 therefore provides a two-locus control of T cell activation and proliferation, (1) by blocking initial signals transmitted via the T cell receptor and (2) by blocking progression of T cell proliferation by blocking IL2-mediated responses.

This selective effect of Lyp1 renders it an ideal target for candidate immunosuppressive drugs which can be used, for example, in organ or tissue graft rejection, graft versus host disease, and autoimmune diseases, including diabetes, rheumatic diseases, multiple sclerosis and other nervous system diseases.

Furthermore, Jak3 kinase activity is of crucial importance in the proliferation of lymphoma cells. Reducing or blocking the activity of Jak3 kinase and Zap70 kinase by manipulating the activity of Lyp1, for example by causing its over-expression, provides a powerful means of reducing or preventing thc growth of T cell lymphomas.

Similarly, Zap70 and Jak3 kinase activities are important in thymocyte differentiation and control of these kinase activities by manipulation of Lyp1 activity provides a method for controlling thymocyte differentiation.

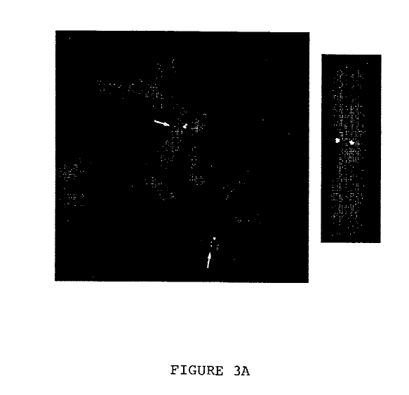

Using FISH analysis, the Lyp gene was found to be localized to chromosome 1p13 (FIG. 3). This region is of particular interest because it is a common site of chromosomal rearrangement in both solid and hematopoietic cancers (47, 48). One such chromosomal rearrangement is a frequent alteration in the 13 region in chromosomally aberrant clones isolated from both patients with Hodgkin's (49, 50) and non Hodgkin's (51) lymphomas. Several lines of evidence already suggest that PTPases may act as tumour suppression genes (2, 52). This suggests an association between an abnormality of the 1p13 locus in these patients and an alteration of Lyp and thus an involvement of Lyp in tumorigenesis.

Isolated Nucleic Acids

In accordance with one series of embodiments, this invention provides isolated polynucleotides corresponding to the nucleotide sequences encoding the human Lyp1 and Lyp2 proteins. The polynucleotides may be in the form of DNA, genomic DNA, cDNA or mRNA or an anti-sense DNA corresponding to a disclosed sequence. Also provided are portions of the Lyp sequences useful as probes and PCR primers or for encoding fragments, functional domains or antigenic determinants of Lyp proteins.

One of ordinary skill in the art is now enabled to identify and isolate Lyp genes or cDNAs which are allelic variants of the disclosed Lyp sequences, using standard hybridization screening or PCR techniques.

Depending on the intended use, the invention provides portions of the disclosed nucleotide sequences comprising about 10 consecutive nucleotides (e.g. for use as PCR primers) to nearly the complete disclosed nucleotide sequences. The invention provides isolated nucleotide sequences comprising sequences corresponding to at least 10, preferably 15 and more preferably at least 20 consecutive nucleotides of the Lyp gene as disclosed or enabled herein or their complements.

In addition, the isolated polynucleotides of the invention include any of the above described nucleotide sequences included in a vector.

In accordance with a further embodiment, the invention provides an isolated polynucleotide which encodes a Lyp protein having an amino acid sequence of greater than 70% overall identity to the amino acid sequence of Table 2 or 4.

The invention also includes polynucleotides which are complementary to the disclosed nucleotide sequences, polynucleotides which hybridize to these sequences under high stringency and degeneracy equivalents of these sequences.

In accordance with a further embodiment, the invention also includes an isolated polynucleotide which encodes a Lyp protein having an amino acid sequence with at least 80% overall identity, preferably at least 90% overall identity to the amino acid sequence of Table 2 or 4.

Proteins

This invention provides Lyp proteins and a method for producing such proteins.

In accordance with one embodiment, a Lyp protein has an amino acid sequence having greater than 70% overall identity to the amino acid sequence of Lyp1 (Table 2, Sequence ID No:2).

In accordance with a further embodiment, a Lyp protein has an amino acid sequence having at least 80% overall identity, preferably at least 90%, to the amino acid sequence of Lyp1.

In accordance with a further embodiment, the invention provides substantially purified Lyp proteins, including the proteins of Table 2 (Lyp1) and Table 4 (Lyp2).

The invention includes analogs of the disclosed protein sequences, having conservative amino acid substitutions therein. The invention also includes fragments of the disclosed protein sequences, such as peptides comprising at least 5, preferably 10 and more preferably 20 consecutive amino acids of the disclosed protein sequences.

The invention further provides polypeptides comprising at least one functional domain or at least one antigenic determinant of a Lyp protein.

In accordance with a further embodiment, the invention enables the production of Lyp proteins, such as human Lyp1 and Lyp 2.

Lyp proteins may be produced by culturing a host cell transformed with a DNA sequence encoding a selected Lyp protein. The DNA sequence is operatively linked to an expression control sequence in a recombinant vector so that the protein may be expressed.

Host cells which may be transfected with the vectors of the invention may be selected from the group consisting of E. coli, Pseudomonas, Bacillus subtillus, or other bacilli, yeasts, fungi, insect cells or mammalian cells including human cells.

For transformation of a mammalian cell for expression of a Lyp protein, the vector may be delivered to the cells by a suitable vehicle. Such vehicles including vaccinia virus, adenovirus, retrovirus, Herpes simplex virus and other vector systems known to those of skill in the art.

A Lyp protein may also be recombinantly expressed as a fusion protein. For example, the Lyp cDNA sequence is inserted into a vector which contains a nucleotide sequence encoding another peptide (e.g. GST-glutathione succinyl transferase). The fusion protein is expressed and recovered from prokaryotic (e.g. bacterial or baculovirus) or eukaryotic cells. The fusion protein can then be purified by affinity chromatography based upon the fusion vector sequence and the Lyp protein obtained by enzymatic cleavage of the fusion protein.

The protein may also be produced by conventional chemical synthetic methods, as understood by those skilled in the art.

Lyp proteins may also be isolated from cells or tissues, including mammalian cells or tissues, in which the protein is normally expressed.

The protein may be purified by conventional purification methods known to those in the art, such as chromatography methods, high performance liquid chromatography methods or precipitation.

For example, an anti-Lyp antibody may be used to isolate a Lyp protein which is then purified by standard methods.

To produce Lyp proteins recombinantly, for example E. coli can be used using the T7 RNA polymerase/promoter system using two plasmids or by labeling of plasmid-encoded proteins, or by expression by infection with M13 Phage mGPI-2. E. coli vectors can also be used with Phage lamba regulatory sequences, by fusion protein vectors (eg. lacZ and trpE), by maltose-binding protein fusions, and by glutathione-S-transferase fusion proteins.

Alternatively, the Lyp1 or Lyp2 protein can be expressed in insect cells using baculoviral vectors, or in mammalian cells using vaccinia virus. For expression in mammalian cells, the cDNA sequence may be ligated to heterologous promoters, such as the simian virus (SV40) promoter in the pSV2 vector and introduced into cells, such as COS cells to achieve transient or long-term expression. The stable integration of the chimeric gene construct may be maintained in mammalian cells by biochemical selection, such as neomycin and mycophoenolic acid.

The Lyp DNA sequence can be altered using procedures such as restriction enzyme digestion, fill-in with DNA polymerase, deletion by exonuclease, extension by terminal deoxynucleotide transferase, ligation of synthetic or cloned DNA sequences, site-directed sequence alteration with the use of specific oligonucleotides together with PCR.

The cDNA sequence or portions thereof, or a mini gene consisting of a cDNA with an intron and its own promoter, is introduced into eukaryotic expression vectors by conventional techniques. These vectors permit the transcription of the cDNA in eukaryotic cells by providing regulatory sequences that initiate and enhance the transcription of the cDNA and ensure its proper splicing and polyadenylation. The endogenous Lyp gene promoter can also be used. Different promoters within vectors have different activities which alters the level of expression of the cDNA. In addition, certain promoters can also modulate function such as the glucocorticoid-responsive promoter from the mouse mammary tumor virus.

Some of the vectors listed contain selectable markers or neo bacterial genes that permit isolation of cells by chemical selection. Stable long-term vectors can be maintained in cells as episomal, freely replicating entities by using regulatory elements of viruses. Cell lines can also be produced which have integrated the vector into the genomic DNA. In this manner, the gene product is produced on a continuous basis.

Vectors are introduced into recipient cells by various methods including calcium phosphate, strontium phosphate, electroporation, lipofection, DEAE dextran, microinjection, or by protoplast fusion. Alternatively, the cDNA can be introduced by infection using viral vectors.

Eukaryotic expression systems can be used for many studies of the Lyp gene and gene product(s) including determination of proper expression and post-translational modifications for full biological activity, identifying regulatory elements located in the 5′ region of the Lyp gene and their role in tissue regulation of protein expression, production of large amounts of the normal and mutant protein for isolation and purification, to use cells expressing the Lyp1 or Lyp2 protein as a functional assay system for antibodies generated against the protein or to test effectiveness of pharmacological agents, or as a component of a signal transduction system, to study the function of the normal complete protein, specific portions of the protein, or of naturally occurring and artificially produced mutant proteins.

Using the techniques mentioned, the expression vectors containing the Lyp1 or Lyp2 cDNA sequence or portions thereof can be introduced into a variety of mammalian cells from other species or into non-mammalian cells.

The recombinant cloning vector, according to this invention, comprises the selected DNA of the DNA sequences of this invention for expression in a suitable host. The DNA is operatively linked in the vector to an expression control sequence in the recombinant DNA molecule so that Lyp1 or Lyp2 protein can be expressed. The expression control sequence may be selected from the group consisting of sequences that control the expression of genes of prokaryotic or eukaryotic cells and their viruses and combinations thereof. The expression control sequence may be selected from the group consisting of the lac system, the trp system, the tac system, the trc system, major operator and promoter regions of phage lambda, the control region of the fd coat protein, early and late promoters of SV40, promoters derived from polyoma, adenovirus, retrovirus, baculovirus, simian virus, 3-phosphoglycerate kinase promoter, yeast acid phosphatase promoters, yeast alpha-mating factors and combinations thereof.

Expression of the Lyp gene in heterologous cell systems may also be used to demonstrate structure-function relationships as well as to provide cell lines for the purposes of drug screening. Ligating the Lyp DNA sequence into a plasmid expression vector to transfect cells is a useful method to test the proteins influence on various cellular biochemical parameters including the identification of substrates as well as activators and inhibitors of the phosphatase. Plasmid expression vectors containing either the entire coding sequence for Lyp1 or Lyp2, or for portions thereof, can be used in in vitro mutagenesis experiments which will identify portions of the protein crucial for regulatory function.

The DNA sequence can be manipulated in studies to understand the expression of the gene and its product. The changes in the sequence may or may not alter the expression pattern in terms of relative quantities, tissue-specificity and functional properties.

Antibodies

In accordance with another embodiment, the present invention enables antibodies which bind selectively to a Lyp protein or to an antigenic determinant of a Lyp protein. As used herein with respect to an antibody, an antibody is said to “bind selectively” to a target if the antibody recognises and binds to the target of interest but does not substantially recognise and bind to other molecules in a sample which includes the target of interest.

Generation of antibodies enables the visualization of the protein in cells and tissues using Western blouing. In this technique, proteins are run on polyacrylamide gel and then transferred onto nitrocellulose membranes. These membranes are then incubated in the presence of the antibody (primary), then following washing are incubated to a secondary antibody which is used for detection of the protein-primary antibody complex. Following repeated washing, the entire complex is visualized using colourimetric or chemiluminescent methods.

Antibodies to an Lyp protein also allow for the use of immunocytochemistry and immunofluorescence techniques in which the proteins can be visualized directly in cells and tissues. This is useful to establish the subcellular location of the protein and the tissue specificity of the protein.

Antibodies to an Lyp protein may also be used to inhibit the protein's activity, where reduced activity is desired.

In general, methods for the preparation of antibodies are well known (42). In order to prepare polyclonal antibodies, fusion proteins containing, for example, defined portions or all of the Lyp1 or Lyp2 protein or specific Lyp1 or Lyp2 generated mutants can be synthesized in bacteria by expression of corresponding DNA sequences in a suitable cloning vehicle. The protein can then be purified, coupled to a carrier protein and mixed with Freund's adjuvant (to help stimulate the antigenic response by the rabbits) and injected into rabbits or other laboratory animals. Alternatively, protein can be isolated from cultured cells expressing the protein. Following booster injections at bi-weekly intervals, the rabbits or other laboratory animals are then bled and the sera isolated. The sera can be used directly or purified prior to use, by affinity chromatography. The sera can then be used to probe protein extracts run on a polyacrylamide gel to identify the Lyp1 or Lyp2 protein or mutant protein. Alternatively, synthetic peptides can be made to the antigenic portions of these proteins and used to innoculate the animals.

To produce monoclonal Lyp1 or Lyp2 antibodies, cells actively expressing the protein are cultured or isolated from tissues and the cell extracts isolated. The extracts or recombinant protein extracts, containing the Lyp1 or Lyp2 protein, are injected in Freund's adjuvant into mice. After being injected 9 times over a three week period, the mice spleens are removed and resuspended in phosphate buffered saline (PBS). The spleen cells serve as a source of lymphocytes, some of which are producing antibody of the appropriate specificity. These are then fused with a permanently growing myeloma partner cell, and the products of the fusion are plated into a number of tissue culture wells in the presence of a selective agent such as HAT. The wells are then screened to identify those containing cells making useful antibody by ELISA. These are then freshly plated. After a period of growth, these wells are again screened to identify antibody-producing cells. Several cloning procedures are carried out until over 90% of the wells contain single clones which are positive for antibody production. From this procedure a stable line of clones is established which produce the antibody. The monoclonal antibody can then be purified by affinity chromatography using Protein A Sepharose. Suitable methods for antibody production may be found in standard texts such as Antibody Engineering, 2^{nd} Edition, Barreback, E. D., Oxford University Press (1995).

Transgenic Animal Models

In accordance with a further embodiment, the invention provides non-human transgenic animals and methods for the production of non-human transgenic animals which afford models for further study of Lyp proteins and tools for screening candidate compounds as potential therapeutic agents. For example, knock-out animals such as mice may be produced with deletion of the Lyp gene. These animals may be examined for phenotypic changes and used to screen candidate compounds for effectiveness to reverse these changes.

In general, techniques of generating transgenic animals are widely accepted and practiced. A laboratory manual on the manipulation of the mouse embryo, for example, is available, detailing standard laboratory techniques for the production of transgenic mice (41).

There are several ways in which to create an animal model in which the Lyp gene is expressed. One could simply generate a specific mutation in the mouse Lyp gene as one strategy. Alternatively, a wild type human Lyp gene and/or a humanized murine gene could be inserted into the animals genome by homologous recombination. It is also possible to insert a mutant (single or multiple) human gene as genomic or minigene construct using wild type or mutant or artificial promoter elements. More commonly, and most preferred in the present invention, knock-out of the endogenous murine genes may be accomplished by the insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

In general, for gene knock-out, embryonic stem cells heterozygous for a knockout mutation in a gene of interest (ie. Lyp gene) and homozygous for a marker gene (eg. coat colour) are transplanted into the blastocoel cavity of 4.5 day embryos homozygous for an alternate marker. The early embryos then are implanted into a pseudopregnant female. Some of the resulting progeny are chimeras. Chimeric mice then are backcrossed. Intercrossing will eventually produce individuals homozygous for the disrupted allele that is, knockout mice. (Capecchi, M R. 1989. Science. 244:1299-1291).

To inactivate the Lyp mouse gene, chemical or x-ray mutagenesis of mouse gametes, followed by fertilization, can be applied. Heterozygous offspring can then be identified by Southern blotting to demonstrate loss of one allele by dosage, or failure to inherit one parental allele using RFLP markers.

To create a transgenic mouse, a mutant or normal version of the human Lyp gene can be inserted into a mouse germ line using standard techniques of oocyte microinjection or transfection or microinjection into stem cells. Alternatively, if it is desired to inactivate or replace the endogenous Lyp gene, homologous recombination using embryonic stem cells may be applied.

For oocyte injection, one or more copies of a mutant or normal Lyp gene can be inserted into the pronucleus of a just-fertilized mouse oocyte. This oocyte is then reimplanted into a pseudo-pregnant foster mother. The liveborn mice can then be screened for integrants using analysis of tail DNA for the presence of human Lyp gene sequences. The transgene can be either a complete genomic sequence injected as a YAC or chromosome fragment, a cDNA with either the natural promoter or a heterologous promoter, or a minigene containing all of the coding region and other elements found to be necessary for optimum expression.

Retroviral infection of early embryos can also be done to insert the human Lyp gene. In this method, the human Lyp gene is inserted into a retroviral vector which is used to directly infect mouse embryos during the early stages of development to generate a chimera, some of which will lead to germline transmission.

Homologous recombination using stem cells allows for the screening of gene transfer cells to identify the rare homologous recombination events. Once identified, these can be used to generate chimeras by injection of mouse blastocysts, and a proportion of the resulting mice will show germline transmission from the recombinant line. This methodology is especially useful if inactivation of the human Lyp gene is desired. For example, inactivation of the Lyp gene can be done by designing a DNA fragment which contains sequences from a Lyp exon flanking a selectable marker. Homologous recombination leads to the insertion of the marker sequences in the middle of an exon, inactivating the Lyp gene. DNA analysis of individual clones can then be used to recognize the homologous recombination events.

Screening for Lyp Mutations

In another embodiment of the invention, the knowledge of the human Lyp sequence provides for screening for diseases involving abnormally activated or inactivated Lyp1 or Lyp2 in which the activity defect is due to a mutant Lyp gene. For example, unregulated Jak 3 kinase leads to tumorigenesis (Schwaller, J. et al., (1998), EMBO J., v. 17, p. 5321-33; Lacronique et al., (1997), Science, v. 278, p. 1309-12; Peeters et al., (1997), Blood, v. 90, p. 2535-40). A loss of Lyp activity, for example through a null mutation of Lyp, may lead to tumour formation, for example leukemia. Other defects associated with loss of Lyp function may include autoimmune disorders such as rheumatoid arthritis.

People at risk for a lymphoid disease or, individuals not previously known to be at risk, or people in general may be screened routinely using probes to detect the presence of a mutant Lyp gene by a variety of techniques. Genomic DNA used for the diagnosis may be obtained from body cells, such as those present in the blood, tissue biopsy, surgical specimen, or autopsy material. The DNA may be isolated and used directly for detection of a specific sequence or may be PCR amplified prior to analysis. RNA or cDNA may also be used. To detect a specific DNA sequence hybridization using specific oligonucleotides, direct DNA sequencing, restriction enzyme digest, RNase protection, chemical cleavage, and ligase-mediated detection are all methods which can be utilized. Oligonucleotides specific to mutant sequences can be chemically synthesized and labelled radioactively with isotopes, or non-radioactively using biotin tags, and hybridized to individual DNA samples immobilized on membranes or other solid-supports by dot-blot or transfer from gels after electrophoresis. The presence or absence of these mutant sequences is then visualized using methods such as autoradiography, fluorometry, or colorimetric reaction. Suitable PCR primers can be generated which are useful for example in amplifying portions of the subject sequence containing identified mutations.

Direct DNA sequencing reveals sequence differences between normal and mutant Lyp DNA. Cloned DNA segments may be used as probes to detect specific DNA segments. PCR can be used to enhance the sensitivity of this method. PCR is an enzymatic amplification directed by sequence-specific primers, and involves repeated cycles of heat denaturation of the DNA, annealing of the complementary primers and extension of the annealed primer with a DNA polymerase. This results in an exponential increase of the target DNA.

Other nucleotide sequence amplification techniques may be used, such as ligation-mediated PCR, anchored PCR and enzymatic amplification as would be understood by those skilled in the art.

Sequence alterations may also generate fortuitous restriction enzyme recognition sites which are revealed by the use of appropriate enzyme digestion followed by gel-blot hybridization. DNA fragments carrying the site (normal or mutant) are detected by their increase or reduction in size, or by the increase or decrease of corresponding restriction fragment numbers. Genomic DNA samples may also be amplified by PCR prior to treatment with the appropriate restriction enzyme and the fragments of different sizes are visualized under UV light in the presence of ethidium bromide after gel electrophoresis.

Genetic testing based on DNA sequence differences may be achieved by detection of alteration in electrophoretic mobility of DNA fragments in gels. Small sequence deletions and insertions can be visualized by high resolution gel electrophoresis. Small deletions may also be detected as changes in the migration pattern of DNA heteroduplexes in non-denaturing gel electrophoresis. Alternatively, a single base substitution mutation may be detected based on differential primer length in PCR. The PCR products of the normal and mutant gene could be differentially detected in acrylamide gels.

Nuclease protection assays (SI or ligase-mediated) also reveal sequence changes at specific locations. Alternatively, to confirm or detect a polymorphism restriction mapping changes ligated PCR, ASO, REF-SSCP and SSCP may be used. Both REF-SSCP and SSCP are mobility shift assays which are based upon the change in conformation due to mutations.

DNA fragments may also be visualized by methods in which the individual DNA samples are not immobilized on membranes. The probe and target sequences may be in solution or the probe sequence may be immobilized. Autoradiography, radioactive decay, spectrophotometry, and fluorometry may also be used to identify specific individual genotypes.

According to an embodiment of the invention, the portion of the DNA segment that is informative for a mutation can be amplified using PCR. The DNA segment immediately surrounding a specific mutation acquired from peripheral blood or other tissue samples from an individual can be screened using constructed oligonucleotide primers. This region would then be amplied by PCR, the products separated by electrophoresis, and transferred to membrane. Labelled probes are then hybridized to the DNA fragments and autoradiography performed.

Drug Screening Methods

In accordance with one embodiment, the invention enables a method for screening candidate compounds for their ability to increase or decrease the phosphatase activity of a Lyp protein. The method comprises providing an assay system for assaying Lyp phosphatase activity, assaying the phosphatase activity in the presence or absence of the candidate compound and determining whether the compound has increased or decreased the control phosphatase activity.

The effect of a candidate compound on Lyp phosphatase activity may be determined, for example, in an assay system such as that described in Example 7 herein.

In accordance with a further embodiment, the invention enables a method for screening candidate compounds for their ability to increase or decrease expression of a Lyp protein. The method comprises contacting a cell with a candidate compound, wherein the cell includes a regulatory region of a Lyp gene operably joined to a coding region, and detecting a change in expression of the coding region.

In one embodiment, the present invention enables culture systems in which cell lines which express the Lyp gene, and thus Lyp1 and/or Lyp2 protein products, are incubated with candidate compounds to test their effects on Lyp expression. Such culture systems can be used to identify compounds which upregulate or downregulate Lyp expression or its function through the interaction with other proteins.

Such compounds can be selected from protein compounds, chemicals and various drugs which are added to the culture medium. After a period of incubation in the presence of a selected test compound(s), the expression of Lyp can be examined by quantifying the levels of Lyp mRNA using standard Northern blotting procedure as described in the examples included herein to determine any changes in expression as a result of the test compound. Cell lines transfected with constructs expressing Lyp can also be used to test the function of compounds developed to modify the protein expression. In addition, transformed cell lines expressing a normal Lyp protein could be mutagenized by the use of mutagenizing agents to produce an altered phenotype in which the role of mutated Lyp can be studied in order to study structure/function relationships of the protein products and their physiological effects.

Alternatively, rather than evaluating the levels of Lyp expression in the presence of a test compound, other proteins which interact with the Lyp protein products may be assessed through phosphorylation assays as are described herein in the examples. Such assays would identify the role of certain compounds on Lyp function and subsequent intracellular protein interaction and physiological effect.

The effect of drugs/agents which intereact with the Lyp protein normal function could be studied in order to more precisely define the intracellular role of Lyp1 and Lyp2 proteins with respect to other proteins. In the present invention, it is demonstrated that Lyp1 down-regulates T cell Zap-70 tyrosine kinase activity and thus activation of T cells. Lyp1 is also demonstrated to down-regulate Lyn kinase activity. This strongly suggests a role for Lyp1 in the regulation of antigen receptor signalling. Therefore, these pathways could be further elucidated by the identification of drugs/agents which alter Lyp1 and thus such antigen receptor signalling and further downstream physiological effects. Such cell culture assays may elucidate the specific nature of Lyp1 in the regulation of the Zap and Lyn family kinases. Incubating cell cultures expressing Lyp with agents that affect phosphorylation may also help to elucidate the involvement of other down stream proteins such as DNA-binding proteins and transcription factors in transcription regulation.

As Lyp is demonstrated to down-regulate the activity of the cytoplasmic Jak3 tyrosine kinase and prevent this kinase from phosphorylating the IRS substrate proteins, cell culture assays as described herein can help to identify candidate compounds to inhibit the effect of Lyp on Jak3 tyrosine kinase activity or modify its effect and thus down-stream intracellular signalling and physiological effects. This may help to identify compounds which regulate activation of cytokine receptors which act through the Jak3 tyrosine kinase signal transduction cascade.

All testing for novel drug development is well suited to defined cell culture systems which can be manipulated to express Lyp and study the result of Lyp protein signalling and gene transcription. Animal models are also important for testing novel drugs and thus may also be used to identify any potentially useful compound affecting Lyp expression and activity and thus physiological function.

Compounds which are found to increase the phosphatase activity of Lyp protein, or to increase expression of Lyp protein, are lead compounds with potential as immunosuppressive agents, for example by reducing or preventing T cell activation. Such immunosuppressive agents can be employed to treat conditions requiring immunosuppression, including autoimmune diseases such as rheumatic diseases, diabetes, and multiple sclerosis and transplant situations, where suppression of graft rejection or graft versus host reactions are required.

Treatment

This invention enables a method for modulating signalling mediated by the T cell receptor, the method comprising administering to a T cell an agent which increases Lyp phosphatase activity or increases Lyp expression in the T cell.

The invention further enables a method for reducing or preventing T cell activation and/or proliferation, the method comprising administering to the T cell an agent which increases Lyp phosphatase activity or increases Lyp expression in the T cell.

The invention further enables a method for treating a disorder which requires immunosuppression, the method comprising administering to the subject in need of treatment an immunosuppression-effective amount of an agent which increases Lyp phosphatase activity or increases Lyp expression.

In accordance with a further embodiment, the invention enables a method for treating lymphoma in a subject, the method comprising administering to the subject an agent which increases Lyp phosphatase activity or increases Lyp expression in an amount effective to reduce or prevent lymphoma cell proliferation.

The invention further provides methods for preventing or treating disorders characterised by an abnormality in the T cell receptor signalling pathway or the IL2-mediated signalling pathway, comprising modulating signalling by administration of an agent which increases or decreases Lyp phosphatase activity or Lyp expression. T cell receptor signalling modulation is useful in disorders such as autoimmune diseases and in transplant situations, as discussed elsewhere herein.

In accordance with another embodiment, the present invention enables gene therapy as a potential therapeutic approach, in which normal copies of the Lyp gene are introduced into patients to successfully code for normal Lyp1 or Lyp2 protein in several different affected cell types. Mutated copies of the Lyp gene in which the protein product is inactivated can also be introduced into patients.

Retroviral vectors can be used for somatic cell gene therapy especially because of their high efficiency of infection and stable integration and expression. The targeted cells however must be able to divide and the expression of the levels of normal protein should be high. The full length Lyp gene can be cloned into a retroviral vector and driven from its endogenous promoter or from the retroviral long terminal repeat or from a promoter specific for the target cell type of interest (such as lymphoid cells).

Other viral vectors which can be used include adeno-associated virus, vaccinia virus, bovine papilloma virus, or a herpesvirus such as Epstein-Barr virus.

Gene transfer could also be achieved using non-viral means requiring infection in vitro. This would include calcium phosphate, DEAE dextran, electroporation, and protoplast fusion. Liposomes may also be potentially beneficial for delivery of DNA into a cell. Although these methods are available, many of these are lower efficiency.

Transplantation of normal genes or mutated genes which code for an inactive Lyp1 or Lyp2 into a targeted affected area of the patient can also be useful therapy for any disorder in which Lyp activity is deficient. In this procedure, a Lyp gene is transferred into a cultivatable cell type such as lymphoid cells, either exogenously or endogenously to the patient. The transformed cells are then injected into the patient.

The invention also provides a method for reversing a transformed phenotype resulting from the excess expression of the Lyp human gene sequence, and/or hyperactivation of a Lyp1 or Lyp2 protein product. Antisense based strategies can be employed to explore gene function, inhibit gene function and as a basis for therapeutic drug design. The principle is based on the hypothesis that sequence specific suppression of gene expression can be achieved by intracellular hybridization between mRNA and a complementary anti-sense species. It is possible to synthesize anti-sense strand nucleotides that bind the sense strand of RNA or DNA with a high degree of specificity. The formation of a hybrid RNA duplex may interfere with the processing/transport/translation and/or stability of a target mRNA.

Hybridization is required for an antisense effect to occur. Antisense effects have been described using a variety of approaches including the use of antisense oligonucleotides, injection of antisense RNA, DNA and transfection of antisense RNA expression vectors.

Therapeutic antisense nucleotides can be made as oligonucleotides or expressed nucleotides. Oligonucleotides are short single strands of DNA which are usually 15 to 20 nucleic acid bases long. Expressed nucleotides are made by an expression vector such as an adenoviral, retroviral or plasmid vector. The vector is administered to the cells in culture, or to a patient, whose cells then make the antisense nucleotide. Expression vectors can be designed to produce antisense RNA, which can vary in length from a few dozen bases to several thousand.

Antisense effects can be induced by control (sense) sequences. The extent of phenotypic changes are highly variable. Phenotypic effects induced by antisense are based on changes in criteria such as biological endpoints, protein levels, protein activation measurement and target mRNA levels.

Multidrug resistance is a useful model for the study of molecular events associated with phenotypic changes due to antisense effects since the MDR phenotype can be established by expression of a single gene mdr1 (MDR gene) encoding P-glycoprotein (a 170 kDa membrane glycoprotein, ATP-dependent efflux pump).

In the present invention, mammalian cells in which the Lyp human cDNA has been transfected and which express an abnormal phenotype, can be additionally transfected with anti-sense Lyp (Lyp1 or Lyp2) nucleotide DNA sequences which hybridize to the Lyp gene in order to inhibit the transcription of the gene and reverse or reduce the abnormal phenotype. Alternatively, portions of the Lyp gene can be targeted with an anti-sense Lyp sequence specific for the kinase domains or the unique amino terminal sequence which may be responsible for the malignant phenotype. Expression vectors can be used as a model for anti-sense gene therapy to target the Lyp which is expressed in abnormal cells. In this manner abnormal cells and tissues can be targeted while allowing healthy cells to survive. This may prove to be an effective treatment for cell abnormalities induced by Lyp1 or Lyp2.

Immunotherapy is also possible for the treatment of diseases associated with excess Lyp activity. Antibodies can be raised to a hyperactive Lyp1 or Lyp2 protein (or portion thereof) and then be administered to bind or block the abnormal protein and its deliterious effects. Simultaneously, expression of the normal protein product could be encouraged. Administration could be in the form of a one time immunogenic preparation or vaccine immunization. An immunogenic composition may be prepared as injectables, as liquid solutions or emulsions. The Lyp protein may be mixed with pharmaceutically acceptable excipients compatible with the protein. Such excipients may include water, saline, dextrose, glycerol, ethanol and combinations thereof. The immunogenic composition and vaccine may further contain auxiliarry substances such as emulsifying agents or adjuvants to enhance effectiveness. Immunogenic compositions and vaccines may be administered parenterally by injection subcutaneously or intramuscularly.

The immunogenic preparations and vaccines are administered in such amount as will be therapeutically effective, protective and immunogenic. Dosage depends on the route of administration and will vary according to the size of the host.

### EXAMPLES

The examples are described for the purposes of illustration and are not intended to limit the scope of the invention.

Methods of molecular genetics, protein and peptide biochemistry and immunology referred to but not explicitly described in this disclosure and examples are reported in the scientific literature and are well known to those skilled in the art.

### Example 1

### Isolation of Novel Human Phosphatases, Lyp1 and Lyp2

Thymuses were obtained from children undergoing open heart surgery. Mononuclear cells were isolated by Ficoll-Hypaque gradient. Adherent cells were removed by incubation to plastic dishes for 60 minutes at 37° C. The resulting thymocytes are typically >95% CD3+.

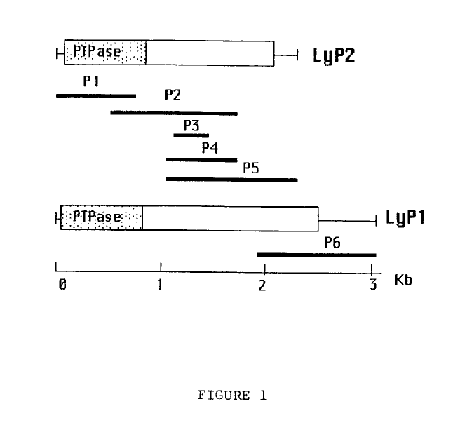

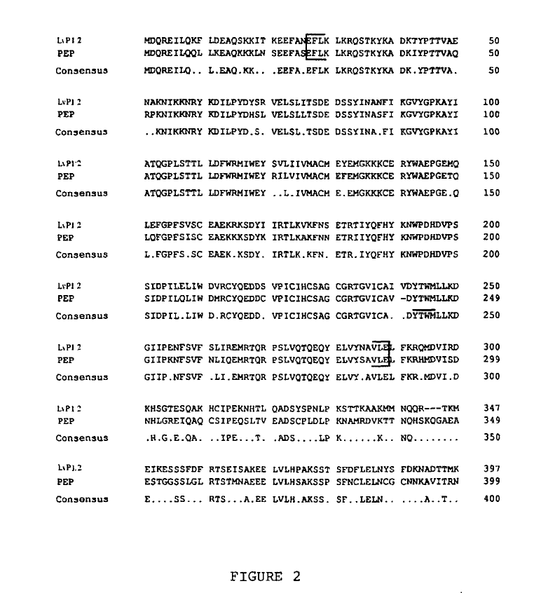

To identify new members of the PTPase gene family that are expressed in thymocytes, a PCT-based approach was used with degenerate oligonucleotides directed at conserved regions of the PTPase catalytic domain. A fragment of 400 bp was amplified from thymocyte cDNA and identified PCR amplified clones corresponding to seven different phosphatases. Six clones were identical to previously isolated human phosphatases:PTP-PEST,²⁴ PTP1B,²⁵ TCPTP,²⁶ HPTPδ,⁶ CD45 and PTPMEG2.²⁷ A seventh clone had no human homologue but was 90% homologous to the murine phosphatase Z70PEP.¹⁰ This clone was used to screen a human thymocyte cDNA library. The first screening isolated two overlapping clones, P1 and P2 (FIG. 1). Clone P2 was used to isolate a further three overlapping clones P3-P5 from the cDNA library. Assembly of the five overlapping clones revealed a single cDNA of 2300 bp containing an open reading frame (ORF) of 2076 bp, predicting a protein of 692 amino acids. The sequence surrounding the putative ATG/methionine start codon contained a purine (A) at position −3 and G at +4, both regarded as important criteria for an eucaryotic initiation site.³² The N-terminal region of the amino acid sequence (FIG. 2) contained a single PTPase catalytic domain characterized by the conserved sequence motif (I/V)HCXXGXXRS/T. This sequence, thought to form the phosphate binding pocket for substrate, is found in all PTPases and is essential for their enzymatic activity. In addition to the 5 overlapping clones a single kb clone was isolated (P6, FIG. 1), with 200 bp of its 5′-end overlapping nucleotides 1950-2055 of the complete cDNA previously isolated. However this was followed by an alternative 700 bp, coding for an ORF totalling 2424 bp. The long (3056 bp) and short (2356 bp) forms share nucleotides 1-2097 but code for alternative C-terminal sequence. These forms are designated Lyp1 and Lyp2 respectively. Lyp2 is an alternative spliced isoform of Lyp1.

Polymerase chain reaction and Subcloning of the Phosphatase Clones.

Total RNA was prepared from thymocytes using Trizol reagent (Gibco-BRL). First strand cDNA synthesis was performed with oligo-dt primer and Superscript 11 RT (Gibco-BRL). This was used as a template for PCR amplification with Taq DNA polymerase (Perkin Elmer Cetus) and the following degenerate primers:

| | | |
|---|---|---|
| PTP1: | GCGGATCCTCIGA(C/T)TA(C/T)AT(A/C/T)AA(T/C)GC | [sense, SEQ. ID NO:5] |
| PTP2: | GCGAATTCCCIACICCIGC(A/G)CT(G/A)CA(G/A)TG. | [antisense, SEQ. ID NO:6] |

These degenerate primers are designed to match two highly conserved sequences within PTPase catalytic domains, XDYINA and HCSAG/VG respectively. PCR was performed as follows: five cycles of 60 sec. at 94° C., 30 sec. at 37° C. and 60 sec. at 72° C., and a further 25 cycles with an annealing temperature of 45° C. Fragments of approximately 400 bp were isolated, cloned and sequenced.

Isolation and Sequencing of Lyp1 and Lyp2 cDNA Clones.

An oligo-dT derived λgt10 cDNA library from human thymocytes was screened with a [³²P]labelled 430 bp Lyp1 fragment obtained by PCR. λPlaques were transferred to ICN Biotrans nylon filters and screened by hybridization at 65° C. in 5×SSC, 5× Denhart's solution, 0.1%SDS (22). Phage DNA was prepared from positive plaques, cDNA inserts were excised, subcloned into pUC-19, and sequenced. To obtain the complete Lyp1 cDNA, secondary and tertiary library screenings were performed with the 1.3 kb and 0.6 kb partial cDNA clones isolated in the first screening (FIG. 1). One clone (P5) from the second screening was found to contain the carboxyterminal sequence of the spliced form of Lyp1 (Lyp2).

### Example 2

### Lyp2 Production by Alternative RNA Splicing of the Lyp1 Message

To confirm the hypothesis that Lyp2 was produced by alternative splicing of Lyp1 RNA, three oligonucleotides matching sequences around the putative splicing sites were used in PCR amplifications on a genomic DNA template (FIG. 4). Oligonucleotide 1 corresponded to the common nucleotides 2076-2097 of Lyp1 and Lyp2 (Table 1 and 2), oligonucleotide 2 to Lyp2 untranslated area adjacent to the stop codon (nucleotides 2150-2168), and olignucleotide 3 to Lyp1 sequence immediately downstream of primer 1 (nucleotides 2098-2120) (FIG. 4A). The resultant PCR products are shown in FIG. 4B. PCR amplification with primers 1 and 3 created an approximately 3.5 kb DNA fragment, suggesting the presence of an intron between the primers. However PCR with primers 1 and 2 resulted in a much smaller fragment of 100 bp, the size expected from Lyp2 cDNA sequence. Upon sequencing, the 5′ end sequence of the 3.5 kb fragment was found to contain the alternative C-terminus, stop codon and untranslated nucleotide sequence of Lyp2 (FIG. 4C). This clearly demonstrated that Lyp1 and Lyp2 are the alternatively spliced transcripts of a single gene. While the 3.5 kb intron is spliced out of the Lyp1 form, this does not occur in the Lyp2 isoform and as a result only 7 amino acids are added and an alternative stop codon is utilized.

### Example 3

### Characterization of Lyp1 and Lyp2 Proteins

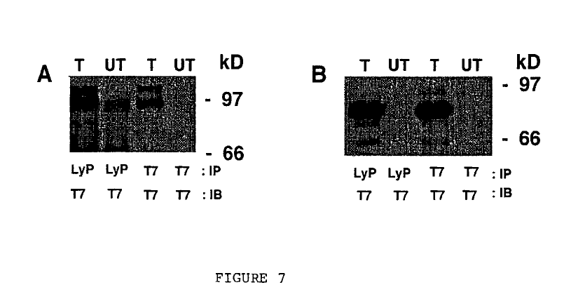

To determine whether Lyp1 and Lyp2 proteins are expressed at their predicted sizes or undergo processing in eukaryotic cells, the full length cDNAs were tagged at their 5′ end with a haemagglutinin (HA) epitope and transfected into COS-7 cells. The cDNAs of Lyp1 and Lyp2 code for polypeptides of molecular weight (Mw) 92,000 and 78,000 respectively. On SDS-PAGE gel the molecular weights of the transfected proteins were close to the predicted values (FIG. 7). Antibodies to the HA tag recognized a single protein with an apparent Mw of 96 kDa in Lyp1 transfected cells and a single protein with an apparent molecular Mw of 80 kDa in Lyp2 transfectants, indicating that these phosphatases do not undergo significant post translation modifications.

Determination of Actual Size of Lyp1 and Lyp2 Proteins

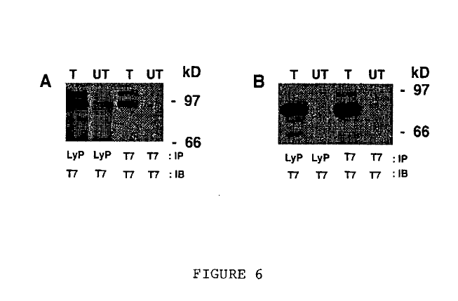

To determine the actual size of the Lyp1 and Lyp2 proteins, the full length cDNAs were cloned by PCR from oligo-dT selected mRNA, tagged with a T7 epitope and transfected into COS-7 cells. The deduced amino acid sequences of Lyp1 and Lyp2 predict molecular weights of 92 kD and 78 kD respectively. Immunoprecipitation of the transfected proteins with anti-T7 or anti-Lyp antibodies and blotting with the T7 antibody showed the protein Lyp2 to have an apparent molecular weight of 85 kD, slightly higher than the predicted molecular weight. Two proteins with apparent molecular weights of 96 kD and 105 kD were observed in COS-7 cells transfected with the Lyp1 cDNA (FIG. 6). Both of these proteins were recognized by the T7 and Lyp antibodies. The lower molecular weight product probably represents the result of proteolytic degradation while the 105 kD protein is intact Lyp1. When immunoprecipated from lymphoid cells lines the native Lyp1 protein has an apparent molecular weight of 105 kD, in agreement with the size observed in transfected COS-7 cells (FIG. 7).

Preferential Lymphoid Expression of Lyp1 Transcripts Cell Preparation and Cell lines Lymphocytes were isolated from tonsil tissue or from peripheral blood of healthy volunteers by Ficoll-Hypaque centrifugation, following by rosetting with neuramimidase treated sheep red blood cells (RBC) to isolate T lymphocytes. After isolating roscttes by Fico11Hypaque gradient centrifugation, T cells were released with ACT treatment (0.75% NH₄Cl in 20 mmol/L Tris, pH 7.2) of the roscttcs to lysc the red blood cells. The buffy layer, containing the B cells, was washed three times with PBS. The resultant T lymphocytes are typically 98% to 99% CD3+ and the B lymphocytes are typically 97% to 98.5% CD 19+.

To induce activation and maturation of peripheral T lymphocytes, 25×10⁶ T cells were stimulated with 2.5 μg/ml of anti-CD3 (Calbiochem) or 10 μg/ml of phytohemagglutinin (PHA) (Gibco BRL) for 24 to 48 hours at 37° C. in RPMI (10% FCS).

Northern blot analysis of mRNA from various human tissues using a Lyp cDNA probe common to both Lyp isoforms revealed a major transcript of approximately 4.4 kb in all of the lymphoid tissues examined (FIG. 5). Substantial levels of Lyp mRNA were detected in spleen, thymus, lymph node, peripheral leukocytes, tonsil B and T lymphocytes, and to a lesser degree in bone marrow. In contrast, Lyp transcripts were not detected in prostate, ovary, testis fetal liver or colon tissues (or other human tissues including heart, lung, brain, placenta, or liver, data not shown). A low level of Lyp expression could however be detected in the small intestine and appendix mucosa, presumably due to the presence of contaminating lymphocytes. Lyp2 expression could not detected by Northern blot analysis using a probe to the last 21 specific nucleotides. Therefore its expression, relative to Lyp1, was quantified by competitive PCR on polyA+-derived single strand thymocyte cDNA. The internal standards were constructed by deleting 140 bp from both Lyp1 and Lyp2 cDNAs. Co-amplification of the target cDNA with various concentrations of internal control revealed that a concentration of 0.05×10⁻⁴ pM control DNA was needed to produce equivalent amounts of Lyp2 target and control PCR product, while 5×10⁴ PM of internal standard was required with specific primers to Lyp1 (FIG. 8). These results suggest that the level of Lyp1 expression in thymocytes is approximately 100 fold greater than the level of Lyp2 expression.

From its expression pattern in normal human tissues and cells, Lyp appears to be a predominantly lymphoid phosphatase, although a low level of expression could also be detected in the monocyte cell line, U937. Myeloid (OCI/AML3: origin and properties previously described (55)) and erythroleukemia (K562) cell lines displayed little to no expression.

Northern Blot Analysis of Lyp1 and Lyp2 RNA.

To further characterize the expression of the Lyp isoforms, Northern blots were performed with a Lyp2 specific cDNA probe on human mRNA from lymphoid and hematopoietic tissues. This revealed a single 5.2 kb transcript in all of the tissues examined, with the highest level of expression in fetal liver (FIG. 5). Subsequennt blotting of the same membrane with a Lyp1 specific probe revealed the dominant 4.4 kb transcript previously observed. Lyp1 demonstrated a high level of expression not only in the mature lymphoid tissues, but also in the thymus. In contrast to Lyp2, Lyp1 mRNA could not be detected in fetal liver and only a low level of expression could be seen in bone marrow.

For the actual northern blotting procedure, total RNA was extracted from thymocytes using Trizol reagent (Gibco BRL). Poly, A+ RNA was isolated by two passages through an oligo(dt) column. 2 μg of Poly A+ RNA per sample was electrophoresed in a 1% agarose formaldehyde gel and capillary blotted onto nitrocellulose filters. Filters and a human multiple tissue poly A+ RNA northern blots (Clontech) were hybridized overnight at 42° C. with [³²P] labelled Lyp cDNA probes in 50% formamide, 5×SSC, 5× Denhart's solution, 0.1% SDS, 50 μl Na₂HPO₄ pH 6.5, and denatured Salmon sperm DNA (100 μg/ml). Specifically, 2 μg of poly A⁺ RNA from various human tissues was hybridized with a 1.3 kb cDNA probe common to both Lyp1 and Lyp2 and exposed 7 days or 24 hr with Actin. After hybridization, the final wash was performed in 0.2%SSC, 0.1% SDS at 55° C. (22).

Relative Quantification of Lyp1 and Lyp2 mRNA by Competitive Polymerase Chain Reaction.

The relative levels of Lyp1 and Lyp2 messenger RNA (mRNA) in thymocytes were quantified by competitive PCR using a synthetic cDNA as internal standard. This technique involves co-amplification of a target cDNA (produced from the corresponding mRNA by reverse transcription) and of the internal standard. The target cDNA and the internal standard use the same primer sequence, but yield PCR products of different sizes that can be resolved on gel electrophoresis. In the exponential phase of the amplification, the amount of target cDNA can be quantified by comparison with the amplification of various amounts of the internal standard. The amount of target sequence in the sample is estimated by the amount of control producing an equivalent amounts of PCR products. The internal standards were constructed by deleting 140 bp from both Lyp1 and Lyp2 cDNAs, using two EcoRI sites found in position 1805 and 1945. PCR primers: The 5′ primer for both Lyp1 and Lyp2-corresponds to nucleotides 1660-1682 with the 3′ primer for LyP1-corresponding to nucleotides 2425-2447, while the 3′ primer for Lyp2-corresponds to nucleotides 2075-2097. cDNA was prepared from oligo (dT) selected mRNA as described previously. Aliquots of thymus cDNA were co amplified with varying amounts of internal standard for 26 cycles for Lyp 1 and 35 cycles for Lyp2. (denaturating 94° C. 30 sec., annealing at 54° C. and elongation 45 sec. at 72° C.). The PCR products (40p1) were electrophoresed on 1.2% agarose gel, stained with ethidium bromide and photographed. The possibility of genomic DNA contamination in the RT PCR reaction was excluded with the appropriate controls.

### Example 4

### Cellular Localization of Lyp1 and Lyp2 in Transfected COS-7 Cells

In order to determine the cellular localization of the two phosphatases, the distribution of both Lyp1 and Lyp2 was determined by indirect immunofluorescence in transiently transfected COS-7 cells. Lyp1 and Lyp2 were inserted into the pcDNA3 eucaryotic expression vector (Invitrogen) and a T7 tag or HA epitope (YPYDVPDYA), as a three-tandem repeat, inserted at the 5′ end of the coding sequences of both Lyp1 and Lyp2 cDNAs. Constructs were verified by sequencing. COS-7 cells were transfected with 2 μg DNA and 17 μl of Lipofectamine for 5 hours, incubated on sterile cover slips in six well plates (0.3×10⁶/plate) in DMEM containing 10% fetal calf serum and stained 48 hours post transfection. The COS-7 cells were then washed in PBS and fixed for 30 min at room temperature in 2% paraformaldehyde. Cell permeabilization was performed with 0.1% Triton X100 and after blocking non-specific sites with 5% donkey serum, the cells were incubated with monoclonal anti-HA (1:1000) from Bico-Berkely, for 60 min at room temperature. The cells were washed and exposed for 45 min to cy3 conjugated affinipure Donkey anti-mouse IgG (1:1000 in PBS) from Jackson Immunoresearch Laboratories Inc. After 3 to 4 washes, immunoreactivity was detected by fluorescence microscopy. COS-7 cells transfected with either Lyp1 and Lyp2 displayed prominent perinuclear and cytoplasmic staining but no staining of the nucleus (FIG. 9). No fluorescence was noted in COS-7 cells transfected with vector alone. The pattern of staining suggests that both of these phosphatases are predominantly cytoplasmic.

Transfection

To examine the actual size of the expressed proteins Lyp1 and Lyp2, cDNAs were inserted into the pcDNA3 eukaryotic expression vector (Invitrogen). An HA epitope (YPYDVPDYA) derived from the haemagglutinin protein of influenza virus, was inserted as three-tandem repeat at the 5′ end of the coding sequences of both Lyp forms. The constructs were verified by sequencing. COS-7 cells (0.5×10⁶) were transfected with 5 μg plasmid DNA in 50 μl of Lipofectamine (Gibco-BRL) for 5 hours according to the manufacture's instructions. 24 hours before transfection 0.5×10⁶ COS-7 cells were plated on 60 mm plates in Dulbaco's modified Eagle medium (DMEM) containing 10% fetal calf serum. To examine the cellular localization of the expressed proteins, Cos-7 cells were transfected with 2 μg DNA and 17 μl of Lipofectamine for 5 hours, incubated on sterile cover slips in six well plates (0.3×10⁶/plate) in DMEM containing 10% fetal calf serum for 48 hours and stained. 48 hours post transfection the COS-7 cells were harvested and solubilized in cold lysis buffer (20 mM Tris pH 7.5, 150 mM NaCl, 1 mM EDTA, 1% NP 40 and 1 mM PMSF).

Immunoprecipitation and Western Blotting

For NP-40 cells, 1% NP40 cell lysates were pre-cleared by centrifugation. Immunoprecipitation of T7 tagged Lyp was carried out by the addition of 11 g of T7 antibody, or by the addition of 5 μl of the Lyp anti-serum followed by the addition of 20 μl of a 50:50 suspension of protein G sepharose (Pharmacia) and incubation overnight at −4° C. Immunoprecipitates were washed three times with lysis buffer and separated by 6% SDS-PAGE. The separated proteins were electrophoretically trasferred to Hybond C Super nitro-cellulose membrane (Amersham Life Science). Membranes were blocked with 5% non-fat milk and blotted with anti-T7 (1:10,000) or with anti Lyp (1:800). Detection was performed with horseradish peroxidase conjugated second antibodies from Amersham Life Science and chemiluminescence reagent from Kirkeggard & Perry Laboratories.

For COS-7 cells, the cells (0.5×10⁻⁶/plate) were washed three times with cold PBS and solubilized in cold lysis buffer (20 mM Tris pH 7.5, 150 mM NaCl, 1 mM EDTA, 1% NP-40, 1 mM PMSF). The lysates were cleared by centrifugation. SDS sample buffer was added to the clarified lysates and resolved by 7% SDS-PAGE. The proteins were electrophoretically transferred to Hybond-C super nitrocellulose membrane (Amersham Life Science). Membranes were blocked with 5% non fat milk and probed with HA monoclonal antibody from Blco-Berkely. Detection was performed with horseradish peroxidase conjugated sheep anti mouse purchased from (Amersham Life Science) and chemiluminescence reagent from Kirkeggard & Perry laboratories.

Indirect Immunofluorescence

48 hours post transfection Cos-7 cells were washed in PBS and fixed for 30 min at room temperature in 2% paraformaldehyde. Cell permeabilization was performed with 0.1% Triton X100. After blocking non-specific sites with 5% Donkey serum, the cells were incubated for 60 min at room temperature with monoclonal anti HA tag (1:1000 in PBS) from Blco-Berkely. The cells were washed and exposed for 45 min. to cy3 conjugated affinipure Donkey anti mouse IgG (1:1000 in PBS) from Jackson Immunoresearch Laboratories, Inc. After 34 washes immunoreactivity was detected by fluorescence microscopy.

### Example 5

### Characterization of Lyp Protein Expression Cell Lines

The G2 pre-preB cell line was derived from a patient with acute lymphocytic leukemia (56). All of the other cell lines used for the present invention were obtained from the American Type Culture Collection. All cells were maintained in RPMI 1640 containing 10% fetal calf serum.

Antibodies

Rabbit polyclonal antibodies were raised to a mixture of two peptides of Lyp, with the amino acid sequences RTKSTPFELIQQR and SKMSLDLPEKQDG. These peptides were chosen from a potentially exposed area, as predicted by Hopp and Woods, in the non-catalytic domain. A second polyclonal antibody was raised to a bacterial fusion protein of the catalytic domain of Lyp (Pet vector—Novagen). After careful testing these antibodies were used for immunoprecipitation and western blotting. T7 antibody was purchased from Novagen (WI), anti-cbl, anti-Jak3 and anti-p110 from Santa Cruz Biotech (CA) and anti-phosphotyrosine from U.B.I. (NY).

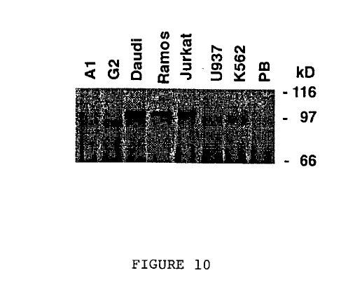

The above-described polyclonal antibodies were first used to characterize the expression of Lyp proteins in human hematopoictic cell lines (FIG. 10). A single band of 105 kD is seeen in both T cell (Jurkat) and B cell lines (Daudi and Ramos), the same size as observed upon transfection of Lyp1 cDNA into COS-7 cells (FIG. 6). Lyp1 expression could not be detected in either the monocytic (U937) or myeloid (K562) cell lines, while low levels of expression could be seen in pre-B cells (G2, A1). This pattern of protein expression correlates with that of Lyp1 mRNA observed by Northern blotting. A protein of the predicted size of Lyp2 (85 kD) in the cell lines examined was not detected.

Expression of the Lyp protein in primary lymphoid cells (FIG. 11) was also examined. Both thymocytes and tonsil T lymphocytes expressed Lyp 1, while resting T cells from peripheral blood, in addition to expressing low levels of Lyp 1, also expressed an 85 kD protein, recognized by both polyclonal Lyp antibodies. This is the predicted molecular weight of Lyp2, the shorter alternatively spliced form of Lypl.

To determine whether expression of the Lyp proteins may be regulated by activation in T cells, normal peripheral blood T lymphocytes were incubated with either PHA, or anti-CD3 and harvested after 24 or 48 hours (FIG. 11B). An increase in the level of Lyp1 protein expression was observed after 24 hours of either stimulus, with a further increase seen after 48 hours with anti-CD3. The 85 kD protein could no longer be detected after a 24 hours incubation with either PHA or anti-CD3.

### Example 6

### Identifying the Chromosomal Location of Lyp

A 1.8 kb Lyp cDNA fragment was used as a probe to examine the chromosomal location of Lyp using fluorescent in situ hybridization. The regional assessment of this cDNA probe was determined by the analysis of 40 well-spread metaphases. Biotynylated Lyp probe was prepared by nick translation for fluorescence in situ hybridization (FISH) to normal human lymphocyte chromosomes (counterstained with propidium iodide and 4′,6-diamidin-3-phenylindol-dihydrochloride, DAPI, according to published methods (43, 44). The probe was detected with avidinfluorescein isothiocyanate (FITC) followed by biotinylated anti-avidin antibody antibody and avidin-FITC. Images of metaphase preparations were captured by thermoelectrically cooled charge coupled camera (Photometrics, Tucson, Ariz.). Separate images of DAPI banded chromosomes (45) and FITC targeted chromosomes were obtained and merged electronically using image analysis software (Yale University, New Haven, Conn.) and pseudo coloured blue (DAPI) and yellow (FITC) as described by Boyle et al., (44). The band assignment was determined by measuring the fractional chromosome length and by analyzing the banding pattern generated by the DAPI counterstained image (46).

Positive hybridization signals at the short arm of human chromosome 1 in region p13 (shown schematically in FIG. 3) were noted in approximately 10% of the cells. The band assignment was determined by measuring the fractional chromosomal length and by analyzing the banding pattern generated by DAPI counterstained image. The low frequency of hybridization obtained with this probe is commonly seen with small cDNA probes of this size. Signals were visualized on both homologues in 90% of the positive spreads (FIG. 3). No fluorescence signal was seen on any other chromosome, impling that the human Lyp gene is located on chromosome 1 in the p13 region.

### Example 7

### Phosphatase Assay

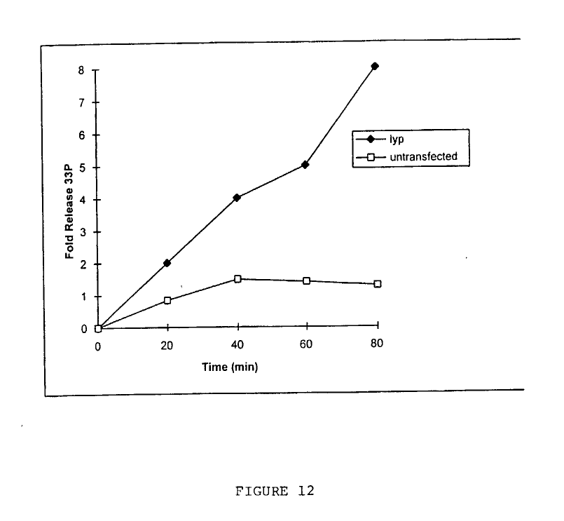

To determine whether Lyp1 possessed a catalytically active tyrosine phosphatase domain, COS cells were transfected with T7-LyP cDNA, the protein immunoprecipitated with anti-T7 and used to dephosphorylate a labelled synthetic peptide, Raytide, in an in vitro phosphatase assay. Raytide peptide was ³³P labelled on tyrosine residues in vitro using the tyrosine kinase p60src and purified on phosphocellulose paper. Release of ³³P over time was measured in the phosphatase assay and compared to controls from untransfected cells. The results showed a seven fold increase in ³³P release from the substrate incubated with Lyp immunoprecipitates compared to control immunoprecipitates (FIG. 12), demonstrating that Lyp does possess tyrosine phosphatase activity. This activity can be completely inhibited by pervanadate (data not shown).

Specifically, the synthetic peptide Raytide was phosphorylated according to the method described by Guan et al., (1994)(54), on tyrosine by p60src (Oncogene Science) as follows: 10 μg Raytide in 50 mM Hepes pH 7.5, 10 mM MgCl₂, 0.067% mercaptoethanol, 0.05 mM ATP was incubated with 300 μCiγ³³P ATP per ml and 2 μg p60src in a final volume of 30 μl. The reaction was allowed to proceed for 30 minutes at 30° C. and was stopped by the addition of 1201 μl 10% phosphoric acid.

The sample was spotted onto two 1×1 cm sheets of P81 phophocellulose paper and extensively washed with 0.5% phosphoric acid. Phosphorylated peptide was eluted twice with 1 ml 500 mM (NH₄)₂CO₃, lyophilized and resuspended in 100 μl H₂O.

The phosphorylated substrate was used in the phosphatase assay as described by Stueli et al (1989)(54). The phosphatase assay mixture, 50 μl, contains 5 μl of ×10 phosphatase (250 mM Hepes pH 7.3, 50 mM EDTA, 100 mM dithiothreitol), 5 μl of radioactive substrate (Raytide) and 5 μl sample (LyP immunoprecipitate) and H₂O to final volume. The assay was allowed to proceed at 30° C. for the indicated time and the reaction terminated by the addition of 750 μl of a charcoal mixture (0.9M HCl, 90 mM sodium pyrophosphate, 2 mM NaH₂PO₄, 4% v/v Norit A). After centrifugation the free ³³P in the supernatant was measured.

### Example 8

### Determination of Involvement of Lyp1 in TCR Signalling

One of the earliest events following TCR stimulation of T cells is the induction of tyrosine phosphorylation. In order to determine whether Lyp played a role in TCR signalling, human thymocytes were stimulated with anti-CD3 for various periods of time, Lyp immunoprecipitated and blotted with anti-phosphotyrosine. This revealed that while Lyp itself is not detectably tyrosine phosphorylated, a heavily phosphorylated protein of 116-120 kD co-precipitates with Lyp, appearing within 1 minute of stimulation (FIG. 13A). Once activated, the phosphorylation level of this protein remained constant over a period of 20 minutes. The 116 kD phosphorylated protein was identified by western blotting of Lyp immunoprecipitates from CD3 stimulated thymocytes with antibodies to various candidate proteins. The 116 kD protein associated with Lyp1 was found to be c-Cbl (FIG. 13B), but not p125Fak, p116 Jak3 or p110 P13-kinase. No alteration in the amount of Cbl co-immunoprecipitating with Lyp could be detected upon anti-CD3 stimulation, suggesting that Lyp1 and Cbl are constitutively associated, although Cbl can be inducibly phosphorylated. This interaction was also observed in the mature T cell line Jurkat (not shown) and further confirmed by transfection of Lyp1 into COS-7 cells and examining its association with the endogenous Cbl protein (FIG. 13C). Lyp1 was found not only to co-precipitate with Cbl in COS cells, but also to reduce significantly the basal level of Cbl tyrosine phosphorylation (FIG. 13D). This suggests that Lyp1 may serve to regulate Cbl function and possibly that of Cbl associated proteins in lymphoid cells.

### Example 9

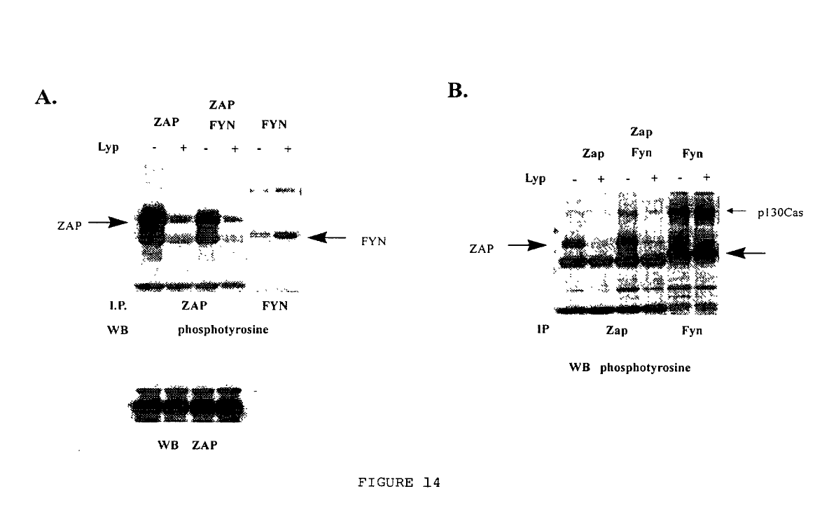

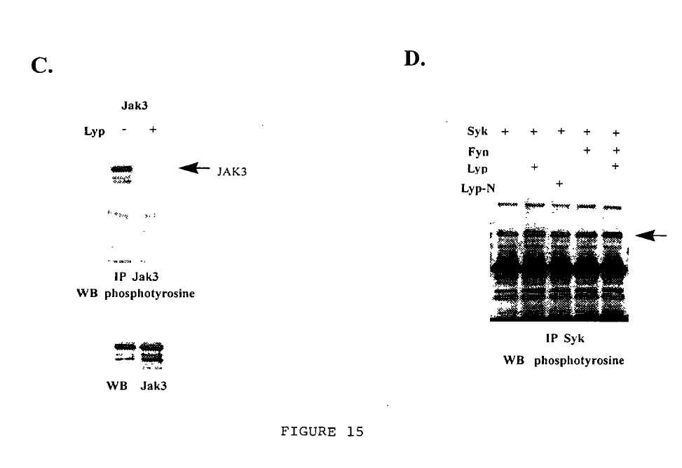

cDNA for the phosphatase Lyp1 and the indicated kinase, in the eucaryotic expression vector pcDNA3, were transiently transfected into either the COS-7 monkey epithelial cell line (A) or the 293T human epithelial cell line as indicated. 48 hours after transfection, cells were harvested, lysates made in 1 & NP-40 lysis buffer and immunoprecipitations performed with antibodies to the transfected kinase. Immunoprecipitates were washed, boiled in SDS sample buffer and electrophoresed on SDS-PAGE. After electro-transfer to nitrocellulose membrane, Western blotting was performed with anti-phosphotyrosine antibodies and chemiluminescent detection reagents. In both COS-7 and 293-T cells, Lyp1 co-transfection clearly resulted in a reduction in Zap-70 phosphorylation while Fyn was unaffected. Lyp1 could also down-regulate Zap-70 after its activation by Fyn in 293-T cells (B, lanes 3 and 4). The closely related Zap family kinase Syk was also unaffected by Lyp1 (C). The results are shown in FIG. 14.

### Example 10

cDNA for the phosphatase Lyp1 and the indicated kinase, in the eucaryotic expression vector pcDNA3, were transiently transfected into the COS-7 monkey epithelial cell line. 48 hours after transfection, cells were harvested, lysates made in 1% NP-40 lysis buffer and immunoprecipitations performed with antibodies to the transfected kinase. Immunoprecipitates were washed, boiled in SDS sample buffer and electrophoesed on SDS-PAGE. After electro-transfer to nitrocellulose membrane, Western blotting was performed with anti-phosphotyrosine antibodies and chemiluminescent detection reagents.

Lyp1 clearly reduced the tyrosine phosphorylation of Jak3 (C), while having little effect upon Syk (D), possibly increasing its phosphorylation slightly; an effect not seen when Syk is co-transfected with a catalytically inactive form of Lyp1 (Lyp-N, where Cysteine 227 is replaced by Serine).

### REFERENCES

1. Brautigan D L: Great expectations: protein tyrosine phosphatases in cell regulation. B. B. A, 1114: 63, 1992.

2. Klein G: The approaching era of the tumor suppressor genes. Science 238:1539, 1987.

3. Denu J M, Stuckey J A, Saper M A, Dixon J E: Form and function in protein dephosphorylation. Cell 87: 361, 1996.

4. Charbonneau H, Tonkes N K: 1002 protein phosphatases. Annu Rev Cell Biol 8:463, 1992.

5. Fischer EH, Charbonneau H, Tonks H K: Protein tyrosine phosphatases: A diverse family of intracellular and transmembrane enzymes. Science 253: 401, 1991.

6. Kruegert N X, Streuli M, Saito H: Structural diversity and evolution of human receptor-like protein tyrosine phosphatases. EMBO J. 9: 3241, 1990.

7. Tonks N K, Neel B G: From form to function: Signaling by protein tyrosine phosphatases. Cell 87: 365, 1996.

8. Brady-Kanlnay S M, Tonks N K: Protein tyrosine phosphatases as adhesion receptors. Curr Opin Cell Biol 7:650, 1995.

9. Frangioni J V, Beahm P H, Shifirin V, Jost C A Neel B G L: The nontransmembrane tyrosine phosphatase PTP-IB locolized to the endoplasmic reticulum via its 35 amino acid C-terminal sequence. Cell 69:545, 1992.

10. Matthews R J, Bowne D B, Flores E, Thomas M L: Characterization of hematopoietic intracellular protein tyrosine phosphatases: Description of phosphatase containing an SH2 domain and another enriched in proline-, glutamic acid-, serine-, and threonine-rich sequences. Mol Cell Biol 12: 2396, 1992.

11. Ware M D, Rosten P, Damen J E, Liu L, Humphries R K, Krystal G: Cloning and characterization of Human SHIP, the 145-kD inositol 5-phosphatase that associates with SHC after cytokine stimulation. Blood 88:2833, 1996.

12. Thomas ML: The leukocyte common antigen family. Annu Rev Immunol 7: 339, 1989.

13. Justement L B, Campbell K S, Chien N C, Cambier J C: Regulation of B cell antigen receptor signal transduction and phosphorylation by CD45. Sceince 252: 1839, 1991.

14. Pingel J T, Thomas M L: Evidence that the leukocyte-common antigen is required for antigen-induced T lymphocyte proliferation. Cell 58: 1055, 1989.

15. Kishihara K, Penninger J, Wallace V A, Kundig T M, Kawai K, Wakeham A, Timms E, Pfeffer K, Ohashi P S, Tomas C F, Christopher J P, Mak T W: Normal B lymphocyte development but impaired T cell maturation in CD45-exon6 protein tyrosine phosphatase-deficiet mice. Cell 74: 143, 1993.

16. Byth K F, Conroy L A, Howlett S, Smith A J H, May J, Alexander D R, Holmes N:CD45-null transgenic mice reveal a positive regulatory role for CD45 in early thymocyte development, in the selection of CD4⁺CDS⁺ thymocytes, and in B cell maturation. J Exp Med 183:1707, 1996.

17. D'Ambrosio D, Hippen K L, Minskoff S A, Mellman I, Pani G, Siminovitch K A, Cambier: Recruitment and activation of PTP1C in negative regulation of antigen receptor signaling by FcBIII Science 268:293, 1995.

18. Yi T, Mui A L, Krystal G, Ihle J N: Hematopoietic cell phosphatase associates with the intreleukin (IL-3) receptor beta chain and down regulates IL-3-induced tyrosine phosphorylation and mitogenesis. Mol Cell Biol 13: 7577, 1993.

19. Raab M, Rudd C E: Hematopoietic cell phosphatase (HCP) regulates p56^{lck} phosphorylation and ZAP-70 binding to T cell receptor chain. B.B.R.C 222:50, 1996.

20. Tsui H W, Siminovitch K A, Souza L, Tsui F W L: Motheaten and viable motheaten mice have mutations in the haematopoietic cell phosphatase gene. Nature Geneties 4:124, 1993.

21. Zanke B, Suzuki H, Kishihara K, Mizzen L, Minden M, Pawson A, Mak T W:Cloning and expression of an inducible lymphoid-specific, protein tyrosine phosphatase (HePTPase). Eur. J. Immunol 22: 253, 1992.

22. Sambrook J, Fristsch E F, Maniatis T: Molecular cloning a laboratory manual, in Irwin N, Ford N, Ferguson M, Ockler M (eds): Cold Spring Harbor laboratory, New York, N.Y., 1989.

23. Laemmli U K: Cleavage of structural proteins during the assembly of the head of bacteriophage T4. Nature 227: 680, 1970.

24. Yang Q, Co D, Sommercom J, Toniks N K: Cloning and expression of PTP-PEST. A novel, human, nonrtransmembrane protein tyrosine phosphatase. J Biol Chem: 268, 1993.

25. Tonks N K, Diltz C D, Fischer E H: Purification of the major protein tyrosine phosphatases of human placenta. J Biol Chem 263: 6722, 1988.

26. Cool D E, Tonks N K, Charbonneau H, Walsh K A, Fischer E H, Krebs E G: cDNA isolated from a human T-cell library encodes a member of the protein-tyrosine-family. Pro Natl Acad Sci USA, 86: 5257, 1989.

27. Gu M, York J D, Warshawsky I, Majerus P W: Identification, cloning, and expression of a cytosolic megakaryocyte protein tyrosine-phosphatase with sequence homology to cytoskeletal protein 4.1 Proc N atl Acad Sci USA 88: 5867, 1991.

28. Kozak M: Point mutation define a sequence flanking the AUG initiator codon that modulates translation by eukaryotic ribosomes. Cell 44: 283, 1986.

29. Laminet A A, Apell G, Conroy L, Kavanaugh W M: Affinity, specificity, and kinetics of the interaction of the SHC phosphotyrosine binding domain with asparagineX-X-phosphotyrosine motifs of growth factor receptors. J Biol Chem 271:264, 1996.

30. Pawson T: Protein modules and sinalling networks. Nature 323:573, 1995.

31. Cohen G B, Ren R, Baltimore D: Modular binding domains in signal transduction proteins. Cell 80: 237, 1995.

32. Cloutier J F, Veillette A: Association of inhibitory tyrosine protein kinase p₅₀CSK with protein tyrosine phosphatase PEP in T cells and other hematopoietic cells. EMBO J. 15:4909, 1996.

33. Rogers S, Wells R, Rechsteiner M: Amino acid sequences common to rapidly degraded proteins: The PEST hypothesis. Science 234: 364, 1986.

34. Loescher P, Pratt G, Rechsteiner M: The C terminus of mouse ornithine decarboxylase confers rapid degradation on dihydrofolate reductase. Support for the pest hypothesis-J Bio Chem 266: 11213, 1991.

35. Flores E, Roy G, Patel D, Shaw A, Thomas M L: Nuclear localization of the PEP protein tyrosine phosphatase. Mol Cell Bio 4: 4938, 1994.

36. Charest A, Wagner J, Shen S H, Tremblay M L: Murine thyrosine Phosphatase-PEST, a stable cytosolic protein tyrosine phosphtase-Biochem J. 308:425, 1995.

37. Nixon P J, Komenda J, Barber J, Deak Z, Vass I, Diner B A: Deletion of the PEST-like region of photosystem two modifies the Q_{B}-binding pocket but does not prevent rapid turnover of D 1. J B'ol Chem 270: 14919, 1995.

38. McLaughlin S, Dixon J E: Alternative splicing gives rise to a nuclear protein tyrosine phoshatase in Drosophila. J Biol Chem 268: 6839, 1993.

39. Reddy R S, Swarup G: Alternative splicing generates four different forms of a nontransmembrane protein tyrosine phosphatase mRNA. DNA Cell Biol 14:1007, 1995.

40. Kamatkar S, Radha V, Nambirajan S, Reddy R S, Swarup G: Two splice variants of thyrosine phosphatase differ in substrate specificity, DNA binding, and subcellular location. J Biol Chem 271: 26755, 1996.

41. Peter M J, Nakagawa M, Doree M, Labbe J C, Nigg E A: Identification of major nucleolar prteins as candidate of cdc2 kinase. Cell 60: 791, 1990.

42. Krek W, Nigg E A: Mutations of p34^{cdc2} phosphorylation sites induce prematuremitotic events in HeLa cells: evidence for a double block to p34cdc²kinase activation in vertebrates. EMBO J. 10: 3331, 1991.

43. Lichter P, Tang C. J., Call K., Hermanson G., Evans G. A., Housman D., Ward D. C. High Resolution Mapping of Human Chromosome 11 by in situ Hybridization with Cosmid Clones. Science. 247: 64, 1990.

44. Boyle A. L., Feltquite D. M., Dracopoli N. C., Housman D. E., Ward D. C. Rapid physical mapping of cloned DNA on banded mouse chromosomes by fluorescence in situ hybridization. Genomics. 12: 106, 1992.

45. Heng H., Tsui I. C. Modes of DAPI banding and simultaneous in situ hybridization. Chromosoma. 102: 325, 1993.

46. Francke U. Digitized and differentially shaded human chromosome idegrams for genomic applications. Cytogene Cell Genet. 65: 206, 1994.

47. Morris S. W., Valentine M. B., Shapiro D. N., Sublett J. E., Deaven L. L., Foust J. T., Robert W. M., Cerretti D. P., Look A. T. Reassignment of the human CSF1 gene to chromosome 1p13-p21. Blood. 78: 2013, 1991.

48. Lion T., Harbott J., Bannier E., Ritterbach J., Jankovic M., Fink F. M., Stojimirovic A., Hermann J., Riehm H. J. Lampert F., Ritter J., Koch H., Gadner H. The translocation t(1;22)(p13;q13) is a nonrandom marker specifically associated with acute megakaryotic leukemia in young children. Blood. 79: 3325, 1992.

49. Schlegelberger B., Weber M. K. Himmier A., Baretels H., Sonnen R., Kuse R., Feller A. C., Grote W. Cytogenetic finding and results of combined immunophenotyping and karyotyping in Hodgkin's disease. Leukemia. 8: 72, 1994.

50. Naumovski L., Utz P. J., Bergstrom S. K., Morgan R., Molina A., Toole J. J., Glader B. E., McFall P., Weiss L. M., Wamke R., Smith S. D. SUP-HD1: a new Hodgkin's disease-derived cell line with lymphoid features produces interferon-gamma. Blood. 74: 2733, 1989.

51. Garry V. F., Danzl T. J., Tarone R., Griffith J., Cervenka J., Krueger L., Whortton E. B., Jr. Nelson R. L. Chromosome rearrangements in fumigant appliers: possible relationship to non-Hodgkin's lymphoma risk. Cancer Epidemiol Biomarkers Prev. 1:287, 1992.

52. Li J., Yen C., Liaw D., Podsypaninia K., Bose S., Wang S. I., Puc J., Miliaresis C., Rodgers L., mcCombie R., Bigner S. H., Giovanella B. C., Ittmann M., Tycko B., Hibshoosh H., Wigler M. H., Parsons R. PTEN, a putative protein tyrosine phosphatase gene mutated in human brain, breast, and prostate cancer. Science. 257: 1943, 1997.

53. Guan K, Broyles S S, and Dixon J E: A Tyr/Ser protein phosphatase encoded by vacinia virus. Nature. 350: 359, 1991.

54. Stueli M, Krueger N X, Tsai A Y M and Saito H: A family of receptor linked protein tyrosine phosphatases in humans and Drosophila. Proc. Nat. Acad. Sci. U.S.A. 86: 3698, 1989.

55. Wang C. Lishner M, Minden M D, McCulloch E A: The effects of Leukemia Inhibitory Factor (LIF) on the blast stem cells of acute mycloblastic leukemia. Leukemia 4: 548, 1990.

56. Kamel-Reid S, Dick J E, Greaves A, Murdoch B, Doedens M, Gunberger T, Thomer P, Freedman M H, Phillips RA, Letarte M: Differential kinetics of engraftment and induction of CD10 on human Pre-B leukemia cell lines in immune deficient scid mice. Leukemia 6:8, 1992.

57. Pan M G, Rim C, Lu K P, Florio T, Stork P J S: Cloning and expression of two structurally distinct receptor-linked protein-tyrosine phosphatases generated by RNA processing from a single gene. J. Biol. Chem. 268: 19284, 1993.

58. Ota Y, Samelson L E: The product of the proto-oncogene c-cbl a negative regulator of the Syk tyrosine kinase. Science 276: 418, 1997.

59. Lupher M L Jr, Songyang Z, Shoelson S E, Cantly L C, Band H: The Cbl phosphotyrosine-binding domain select a D(N/D)XpY motif and bind to the Tyr292 negative regulatory phosphorylating site of ZAP-70. J. Bio. Chem. 272:33140, 1997.

60. Feshchenko E A, Langdon W Y, Tsygankov A Y: Fyn, Yes and Syk Phosphorylation sites in c-Cbl map to the same tyrosine residues that become phosphorylated in activated T cells. J. Biol. Chem. 273: 8323, 1988.

61. Marengere LEM, Mirtsos C, Kozieradzki I, Vellette A, Mak T W, Penninger J M: Proto-oncoprotein Vav interacts with c-Cbl in activated thymocytes and peripheral T cells. J immunol 159: 70, 1997

62. Reedquist K A T, Panchamoorthy F G, Langdon W Y, Shoelson Se, Druker B J, Band H: Stimulation through the T-cell receptor induces Cbl association with Crk proteins and the guanine-nucleotide exchange protein C3G. J Biol Chem 271: 8435, 1996.

63. Smit L, Borst J: The Cbl family of signal transduction molecules: Crit Rev Oncol 8:359, 1997.

TABLE 1

TCCCTCAACCTACTTATAGACTATTTTTCTTGCTCTGCAGCATGGACCAA
AGAGAAATTCTGCAGAAGTTCCTGGATGAGGCCCAAAGCAAGAAAATTAC
TAAAGAGGAGTTTGCCAATGAATTTCTGAAGCTGAAAAGGCAATCTACCA
AGTACAAGGCAGACAAAACCTATCCTACAACTGTGGCTGAGAATGCCAAG
AATATCAAGAAAAACAGATATAAGGATATTTTGCCCTATGATTATAGCCG
GGTAGAACTATCCCTGATAACCTCTGATGAGGATTCCAGCTACATCAATG
CCAACTTCATTAAGGGAGTTTATGGACCCAAGGCTTATATTGCCACCCAG
GGTCCTTTATCTACAACCCTCCTGGACTTCTGGAGGATGATTTGGGAATA
TAGTGTCCTTATCATTGTTATGGCATGCATGGAGTATGAAATGGGAAAGA
AAAAGTGTGAGCGCTACTGGGCTGAGCCAGGAGAGATGCAGCTGGAATTT
GGCCCTTTCTCTGTATCCTGTGAAGCTGAAAAAAGGAAATCTGATTATAT
AATCAGGACTCTAAAAGTTAAGTTCAATAGTGAAACTCGAACTATCTACC
AGTTTCATTACAAGAATTGGCCAGACCATGATGTACCTTCATCTATAGAC
CCTATTCTTGAGCTCATCTGGGATGTACGTTGTTACCAAGAGGATGACAG
TGTTCCCATATGCATTCACTGCAGTGCTGGCTGTGGAAGGACTGGTGTTA
TTTGTGCTATTGTTGATTATACATGGATGTTGCTAAAAGATGGGATAATT
CCTGAGAACTTCAGTGTTTTCAGTTTGATCCGGGAAATGCGGACACAGAG
GCCTTCATTAGTTCAAACGCAGGAACAATATGAACTGGTCTACAATGCTG
TATTAGAACTATTTAAGAGACAGATGGATGTTATCAGAGATAAACATTCT
GGAACAGAGAGTCAAGCAAAGCATTGTATTCCTGAGAAAAATCACACTCT
CCAAGCAGACTCTTATTCTCCTAATTTACCAAAAAGTACCACAAAAGCAG
CAAAAATGATGAACCAACAAAGGACAAAAATGGAAATCAAAGAATCTTCT
TCCTTTGACTTTAGGACTTCTGAAATAAGTGCAAAAGAAGAGCTAGTTTT
GCACCCTGCTAAATCAAGCACTTCTTTTGACTTTCTGGAGCTAAATTACA
GTTTTGACAAAAATGCTGACACAACCATGAAATGGCAGACAAAGGCATTT
CCAATAGTTGGGGAGCCTCTTCAGAAGCATCAAAGTTTGGATTTGGGCTC
TCTTTGTTTGAGGGATGTTCTAATTCTAAACCTGTAAATGCAGCAGGAAG
ATATTTTAATTCAAAGGTGCCAATAACACGGACCAAATCAACTCCTTTTG
AATTGATACAGCAGAGAGAAACCAAGGAGGTGGACAGCAAGGAAAACTTT
TCTTATTTGGAATCTCAACCACATGATTCTTGTTTTGTAGAGATGCAGGC
TCAAAAAGTAATGCATGTTTCTTCAGCAGAACTGAATTATTCACTGCCAT
ATGACTCTAAACACCAAATACGTAATGCCTCTAATGTAAAGCACCATGAC
TCTAGTGCTCTTGGTGTATATTCTTACATACCTTTAGTGGAAAATCCTTA
TTTTTCATCATGGCCTCCAAGTGGTACCAGTTCTAAGATGTCTCTTGATT
TACCTGAGAAGCAAGATGGAACTGTTTTTCCTTCTTCTCTGTTGCCAACA
TCCTCTACATCCCTCTTCTCTTATTACAATTCACATAGTTCTTTATCACT
GAATTCTCCAACCAATATTTCCTCACTATTGAACCAGGAGTCAGCTGTAC
TAGCAACTGCTCCAAGGATAGATGATGAAATCCCCCCTCCACTTCCTGTA
CGGACACCTGAATCATTTATTGTGGTTGAGGAAGCTGGAGAATTCTCACC
AAATGTTCCCAAATCCTTATCCTCAGCTGTGAAGGTAAAAATTGGAACAT
CACTGGAATGGGGTGGAACATCTGAACCAAAGAAATTTGATGACTCTGTG
ATACTTAGACCAAGCAAGAGTGTAAAACTCCGAAGTCCTAAATCAGAACT
ACATCAAGATCGTTGTTCTCCCCCACCTCCTCTCCCAGAAAGAACTCTAG
AGTCCTTCTTTCTTGCCGATGAAGATTGTATGCAGGCCCAATCTATAGAA
ACATATTCTACTAGCTATCCTGACACCATGGAAAATTCAACATCTTCAAA
ACAGACACTGAAGACTCCTGGAAAAAGTTTCACAAGGAGTAAGAGTTTGA
AAATTTTGCGAAACATGAAAAAGAGTATCTGTAATTCTTGCCCACCAAAC
AAGCCTGCAGAATCTGTTCAGTCAAATAACTCCAGCTCATTTCTGAATTT
TGGTTTTGCAAACCGTTTTTCAAAACCCAAAGGACCAAGGAATCCACCAC
CAACTTGGAATATTTAATAAAACTCAGATTTATAATAATATGGGCTGCAA
GTACACCTGCAAATAAAAGTACTAGAATACTGCTAGTTAAAATAAGTGCT
CTATATGCATAATATGAAGATATGCTAATGTGTTAATAGCTTTTAAAAGA
AAAGCAAAATGCCAATAAGTGCCAGTTTTGCATTTTCATATCATTTGCAT
TGAGTTGAAAACTGCAAATAAAAGTTTGTCACTTGAGCTTATGTACAGAA
TGCTATATGAGAAACACTTTTAGAATGGATTTATTTTTCATTTTTGCCAG
TTATTTTTATTTTCTTTTACTTTTCTACATAAACATAAACTCAAAAGGTI
TGTAAGATTTGGATCTCAACTAATTTCTACATTGCCAGAATATACTATAA
AAAGTAAAAAAAAAAACTTACTTTGTGGGTTGCAATACAAACTGCTCTTG
ACAATGACTATTCCCTGACAGTTATTTTTGCCTAAATGGAGTATACCTTG
TAAATCTTCCCAAATGTTGTGGAAAACTGGAATATTAAGAAAATGAGAAA
TTATATTTATTAGAATAAAATGTGCAAATAATGACAATTATTTGAATGTA
ACAAG

TABLE 2

MDQREILQKFLDEAQSKKITKEEFANEFLKLKRQSTKYKADKTYPTFITVAENAKNIKKNRYKDI
LPYDYSRVELSLITSDEDSSYINANFIKGVYGPKAYIATQGPLSTTLLDFWRMIWEYSVLIIVMA
CMEYEMGKKKCERYWAEPGEMQLEFGPFSVSCEAEKRKSDYIIRTLKVKFNSETRTIYQFHYK
NWPDHDVPSSIDPILELIWDVRCYQEDDSVPICIHCSAGCGRTGVICAIVDYTWMLLKDGIIPEN
FSVFSLIREMRTQRPSLVQTQEQYELVYNAVLELFKRQMIDVDKHSGTESQAKHCIPEKNHTL
QADSYSPNLPKSTTKAAKMMNQQRTKMEIKESSSFDFRTSEISAKEELVLHPAKSSTSFDFLEL
NYSFDKNADTTMKWQTKAFPIVGEPLQKHQSLDLGSLLFEGCSNSKPVNAAGRYFNSKVPITR
TKSTPFELIQQRETKEVDSKENFSYLESQPHDSCFVEMQAQKVMHVSSAELNYSLPYDSKHQIR
NASNVKHHDSSALGVYSYIPLVENPYFSSWPPSGTSSKMSLDLPEKQDGTVFPSSLLPTSSTSLF
SYYNSHSSLSLNSPTNISSLLNQESAVLATAPRIDDEIPPPLPVRTPESFIVVEEAGEFSPNVPKSLS
SAVKVKIGTSLEWGGTSEPKKFDDSVILRPSKSVKLRSPKSELHQDRSSPPPPLPERTLESFFLAD
EDCMQAQSIETYSTSYPDTMENSTSSKQTLKTPGKSFTRSKSLKILRNMKKSICNSCPPNKPAES
VQSNNSSSFLNFGFANRFSKPKGPRNPPPTWNI

TABLE 3

TCCCTCAACCTACTTATAGACTAYITITCITGCTCTGCAGCATGGACCAA
AGAGAAATTCTGCAGAAGTTCCTGGATGAGGCCCAAAGCAAGAAAATTAC
TAAAGAGGAGTTTGCCAATGAATTTCTGAAGCTGAAAAGGCAATCTACCA
AGTACAAGGCAGACAAAACCTATCCTACAACTGTGGCTGAGAATGCCAAG
AATATCAAGAAAAACAGATATAAGGATATTTTGCCCTATGATFITATAGC
CGGGTAGAACTATCCCTGATAACCTCTGATGAGGAYFCCAGCTACATCAA
TGCCAACTTCATTAAGGGAGTTTATGGACCCAAGGCTTATATTGCCACCC
AGGGTCCTTTATCTACAACCCTCCTGGACTTCTGGAGGATGATTTGGGAA
TATAGTGTCCTTATCATTGTTATGGCATGCATGGAGTATGAAATGGGAAA
GAAAAAGTGTGAGCGCTACTGGGCTGAGCCAGGAGAGATGCAGCTGGAAT
TTGGCCCTTTCTCTGTATCCTGTGAAGCTGAAAAAAGGAAATCTGATTAT
ATAATCAGGACTCTAAAAGTTAAGTTCAATAGTGAAACTCGAACTATCTA
CCAGTTTCATTACAAGAATTGGCCAGACCATGATGTACCTTCATCTATAG
ACCCTATTGTTGAGCTCATCTGGGATGTACGTTGTTACCAAGAGGATGAC
AGTGTTCCCATATGCATTCACTGCAGTGCTGGCTGTGGAAGGACTGGTGT
TATTTGTGCTATTGTTGATfATACATGGATGTTGCTAAAAGATGGGATAA
TTCCTGAGAACTTCAGTGTTTTCAGTTTGATCCGGGAAATGCGGACACAG
AGGCCTTCATTAGTTCAAACGCAGGAACAATATGAACTGGTCTACAATGC
TGTATTAGAACTATTTAAGAGACAGATGGATGTTATCAGAGATAAACATT
CTGGAACAGAGAGTCAAGCAAAGCATTGTAAACCTGAGAAAAATCACACT
CTCCAAGCAGACTCTTATTCTCCTAATTTACCAAAAAGTACCACAAAAGC
AGCAAAAATGATGAACCAACAAAGGACAAAAATGGAAATCAAAGAATCTT
CTTCCTTTGACTTTAGGACTTCTGAAATAAGTGCAAAAGAAGAGCTAGTT
TTGCACCCTGCTAAATCAAGCACTTCTTTTGACTITCTGGAGCTAAATTA
CAGGACGACAAAAATGCTGACACAACCATGAAATGGCAGACAAAGGCATT
TCCAATAGTTGGGGAGCCTCTTCAGAAGCATCAAAGTTTGGATTTGGGCT
CTCTTTTGTTTGAGGGATGTTCTAATTCTAAACCTGTAAATGCAGCAGGA
AGATATTTTAATTCAAAGGTGCCAATAACACGGACCAAATCAACTCCTTT
TGAATTGATACAGCAGAGAGAAACCAAGGAGGTGGACAGCAAGGAAAACT
TTTCTTATTTGGAATCTCAACCACATGATTCTTGTTTGTAGAGATGCAGG
CTCAAAAAGTAATGCATGTTTCTTCAGCAGAACTGAATTATTCACTGCCA
TATGACTCTAAACACCAAATACGTAATGCCTCTAATGTAAAGCACCATGA
CTCTAGTGCTCTTGGTGTATATTCTTACATACCTTTAGTGGAAAATCCTT
ATTTTTCATCATGGCGTCCAAGTGGTACCAGTTCTAAGATGTCTCTTGAT
TTACCTGAGAAGCAAGATGGAACTGTTTTTCCTTCTTCTCTGTTGCCAAC
ATCCTGTACATCCCTCTTCTCTTATTACAATTCACATAGTTCTTTATCAC
TGAATTCTCCAACCAATATTTCCTCACTAITGAACCAGGAGTCAGCTGTA
CTAGCAACTGCTCCAAGGATAGATGATGAAATCCCCCCTCCACTTCCTGT
ACGGACACCTGAATCATTTATTGTGGTTGAGGAAGCTGGAGAATTCTCAC
CAAATGTTCCCAAATCCTTATCCTCAGCTGTGAAGGTAAAAATTGGAACA
TCACTGGAATGGGGTGGAACATCTGAACCKAGAAATTTGATGACTCTGTG
ATACTTAGACCAAGCAAGAGTGTAAAACTCCGAAGTCCTAAATCAGGTAA
AAATTTCTCTTGGCTTTAGATGACATTTAGCCCTAAGATTGGAAGAATGG
TTCGTTAAGTTTAGAGTAATTCACTTCAGGAAGTTACTGGTCCCATAATA
GCCCAGTATTCATTGATTTATTTCTGGCTTTCCCAGACTAGAAATTTTGT
AAAGAGTCATGGGGGAAGCTAGGGCTAACCAGAAAATAAAATAAAAATAA
TGGGATAAAAAATCGGAACTACTGTTTTCCCCCTAGTCGGAGCACATCCG
G

TABLE 4

MDQREILQKFLDEAQSKKITKEEFANEFLKLKRQSTKYKADKTYPTTVAE
NAKNIKKNRYDKILPYDYSRVELSLITSDEDSSYINANFIKGVYGPKAYI
ATQGPLSTTLLDFWRMIWEYSVLIIVMACMEYEMGKKKCERYWAEPGEMQ
LEFGPFSVSCEAEKRKSDYIIRTLKVKFNSETRTIYQFHYKNWPDHDVPS
SIDPILELIWDVRCYQEDDSVPICIHCSAGCGRTGVICAIVDYTWMLLKD
GIIPENFSVFSLIIEMRTQRPSLVQTQEQYELVYNAVLELFKRQMDVIRD
KHSGTESQAKHCIPEKNHTLQADSYSPNLPKSTTAAKMMNQQRTKMEIKE
SSSFDFRTSEISAKEELVLHPAKSSTSFDFLELNYSFDKNADTTMKWQTK
AFPIVGEPLQKHQSLDLGSLLFEGCSNSKPVNAAGRYFNSKVPITRTKST
PFELIQQRETKEVDSKENFSYLESQPHDSCFVEMQAQKVMHVSSAELNYS
LPYDSKHQIRNASNVKHHDSSALGVYSYIPLVENPYFSSWPPSGTSSKMS
LDLPEKQDGTVFPSSLLPTSSTSLFSYYNSDSSLSLNSPTNISSLLNQES
AVLATAPRIDDEIPPPLPVRTPESFIVVEEAGESPNVPKSLSSAVKVKIG
TSLEWGGTSEPKKFDDSVILRPSKSVKLRSPKSGKNFSWL

We claim:

1. An isolated polynucleotide comprising a nucleotide sequence encoding a Lyp protein.

2. The polynucleotide of claim 1 wherein the polynucleotide is cDNA, DNA, genomic DNA, RNA or mRNA.

3. The polynucleotide of claim 1 wherein the nucleotide sequence encodes a human LyP protein.

4. The polynucleotide of claim 3 wherein the nucleotide sequence is selected from the group consisting of
(a) a nucleotide sequence encoding the amino acid sequence of Table 2 (Sequence ID NO:2), or a splice variant thereof; and
(b) a nucleotide sequence encoding the amino acid sequence of Table 4 (Sequence ID NO:4).

5. The polynucleotide sequence of claim 4 wherein the nucleotide sequence is selected from the group consisting of
(a) the nucleotide sequence of Table 1 (Sequence ID NO:1);
(b) the nucleotide sequence of Table 3 (Sequence ID NO:3);
(c) a nucleotide sequence complementary to the nucleotide sequence of (a) or (b);
(d) a nucleotide sequence which is a degeneracy equivalent of the nucleotide sequence of (a) or (b); and
(e) a nucleotide sequence which hybridises under stringent conditions to a nucleotide sequence of (a) or (b).

6. An isolated polynucleotide which encodes a Lyp protein having an amino acid sequence of greater than 70% overall identity to the amino acid sequence of Table 2.

7. An isolated polynucleotide which encodes a Lyp protein having an amino acid sequence of greater than 70% overall identity to the amino acid sequence of Table 4.

8. An isolated polynucleotide which encodes a Lyp protein having an amino acid sequence with at least 80% overall identity, preferably at least 90% overall identity to the amino acid sequence of Table 2.

9. An isolated polynucleotide which encodes a Lyp protein having an amino acid sequence with at least 80% overall identity, preferably at least 90% overall identity to the amino acid sequence of Table 4.

10. A nucleotide sequence comprising at least 10, preferably 15 and more preferably 20 consecutive nucleotides of Sequence ID NO:1 or Sequence ID NO:3.

11. A recombinant vector comprising a polynucleotide of any of claims 1 to 10.

12. A host cell comprising the recombinant vector of claim 11.

13. A substantially purified Lyp protein.

14. The protein of claim 13 which is a human Lyp protein.

15. The protein of claim 13 wherein the protein comprises an amino acid sequence selected from
(a) the amino acid sequence of Table 2 (Sequence ID NO:2); and
(b) the amino acid sequence of Table 4 (Sequence ID NO:4).

16. A substantially purified protein having an amino acid sequence of greater than 70% overall identity to the amino acid sequence of Table 2.

17. A substantially purified protein having an amino acid sequence of greater than 70% overall identity to the amino acid sequence of Table 4.

18. A substantially purified protein having an amino acid sequence with at least 80% overall identity, preferably at least 90% overall identity, to the amino acid sequence of Table 2.

19. A substantially purified protein having an amino acid sequence with at least 80% overall identity, preferably at least 90% overall identity, to the amino acid sequence of Table 4.

20. A peptide comprising at least 5, preferably 10, more preferably 20 consecutive amino acids of Sequence ID NO:2 or Sequence ID NO:4.

21. A peptide comprising at least one functional domain of a Lyp protein.

22. A peptide comprising at least one antigenic determinant of a Lyp protein.

23. An antibody which binds specifically to a Lyp protein.

24. The antibody of claim 23 which is a monoclonal antibody.

25. The antibody of claim 23 which is a polyclonal antibody.

26. A hybridoma cell that produces the antibody of claim 16.

27. A method for producing a Lyp protein comprising culturing the host cell of claim 12 under conditions in which the Lyp protein is expressed and isolating the Lyp protein therefrom.

28. A method for screening a candidate compound for an ability to increase or decrease the phosphatase activity of a Lyp protein comprising
(a) providing an assay system for assaying Lyp phosphatase activity;
(b) assaying Lyp phosphatase activity in the presence or absence of the candidate compound; and
(c) determining whether the Lyp phosphatase activity was higher or lower in the presence of the candidate compound than in its absence.

29. A method for screening a candidate compound for ability to modulate expression of a Lyp gene comprising
contacting a cell with a candidate compound, wherein the cell includes a regulatory region of a Lyp gene operably joined to a coding region; and
detecting a change in expression of the coding region.

30. A non-human animal wherein a genome of said animal, or of an ancestor thereof, has been modified by a modification selected from the group consisting of:
(a) knockout of a Lyp gene; and
(b) insertion of a polynucleotide encoding a heterologous Lyp gene.

31. The animal of claim 30 wherein the polynucleotide encodes the amino acid sequence of Table 2 (Sequence ID NO:2) or Table 4 (Sequence ID NO:4).

32. The animal of claim 31 wherein the polynucleotide comprises the nucleic acid sequence of Table 1 (Sequence ID NO:1) or Table 3 (Sequence ID NO:3).

33. A pharmaceutical composition comprising an active ingredient selected from the group consisting of:
(a) an isolated nucleotide sequence encoding a Lyp protein;
(b) a substantially purified Lyp protein;
(c) a substantially purified antibody which binds specifically to a Lyp protein
and a pharmaceutically acceptable carrier.

34. A method for treating a subject having a deficiency of Lyp activity comprising administering to the subject an effective amount of an agent selected from the group consisting of:
(a) an isolated nucleotide sequence encoding a Lyp protein;
(b) a substantially purified Lyp protein.

35. A method for modulating signalling mediated by the T cell receptor, the method comprising administering to a T cell an agent which increases Lyp phosphatase activity or increases Lyp expression in the T cell.

36. A method for reducing or preventing T cell activation and/or proliferation, the method comprising administering to the T cell an agent which increases Lyp phosphatase activity or increases Lyp expression in the T cell.

37. A method for treating a disorder which requires immunosuppression, the method comprising administering to the subject in need of treatment an immunosuppression-effective amount of an agent which increases Lyp phosphatase activity or increases Lyp expression.

38. A method for treating lymphoma in a subject, the method comprising administering to the subject an agent which increases Lyp phosphatase activity or increases Lyp expression in an amount effective to reduce or prevent lymphoma cell proliferation.

39. A method for preventing or treating a disorder characterised by an abnormality in the T cell receptor signalling pathway or the IL2-mediated signalling pathway, comprising modulating signalling by administration of an agent which increases Lyp phosphatase activity or Lyp expression.


SEQUENCE LISTING (7 sequences)

SEQ ID NO: 1 - DNA, 3058 bp, Homo sapiens
  CDS (42)..(2465)
tccctcaacctacttatagactatttttcttgctctgcagcatggaccaaagagaaattctgcagaagttcctggatgaggcccaaagcaagaaaattactaaagaggagtttgccaatgaatttctgaagctgaaaaggcaatctaccaagtacaaggcagacaaaacctatcctacaactgtggctgagaatgccaagaatatcaagaaaaacagatataaggatattttgccctatgattatagccgggtagaactatccctgataacctctgatgaggattccagctacatcaatgccaacttcattaagggagtttatggacccaaggcttatattgccacccagggtcctttatctacaaccctcctggacttctggaggatgatttgggaatatagtgtccttatcattgttatggcatgcatggagtatgaaatgggaaagaaaaagtgtgagcgctactgggctgagccaggagagatgcagctggaatttggccctttctctgtatcctgtgaagctgaaaaaaggaaatctgattatataatcaggactctaaaagttaagttcaatagtgaaactcgaactatctaccagtttcattacaagaattggccagaccatgatgtaccttcatctatagaccctattcttgagctcatctgggatgtacgttgttaccaagaggatgacagtgttcccatatgcattcactgcagtgctggctgtggaaggactggtgttatttgtgctattgttgattatacatggatgttgctaaaagatgggataattcctgagaacttcagtgttttcagtttgatccgggaaatgcggacacagaggccttcattagttcaaacgcaggaacaatatgaactggtctacaatgctgtattagaactatttaagagacagatggatgttatcagagataaacattctggaacagagagtcaagcaaagcattgtattcctgagaaaaatcacactctccaagcagactcttattctcctaatttaccaaaaagtaccacaaaagcagcaaaaatgatgaaccaacaaaggacaaaaatggaaatcaaagaatcttcttcctttgactttaggacttctgaaataagtgcaaaagaagagctagttttgcaccctgctaaatcaagcacttcttttgactttctggagctaaattacagttttgacaaaaatgctgacacaaccatgaaatggcagacaaaggcatttccaatagttggggagcctcttcagaagcatcaaagtttggatttgggctctcttttgtttgagggatgttctaattctaaacctgtaaatgcagcaggaagatattttaattcaaaggtgccaataacacggaccaaatcaactccttttgaattgatacagcagagagaaaccaaggaggtggacagcaaggaaaacttttcttatttggaatctcaaccacatgattcttgttttgtagagatgcaggctcaaaaagtaatgcatgtttcttcagcagaactgaattattcactgccatatgactctaaacaccaaatacgtaatgcctctaatgtaaagcaccatgactctagtgctcttggtgtatattcttacatacctttagtggaaaatccttatttttcatcatggcctccaagtggtaccagttctaagatgtctcttgatttacctgagaagcaagatggaactgtttttccttcttctctgttgccaacatcctctacatccctcttctcttattacaattcacatagttctttatcactgaattctccaaccaatatttcctcactattgaaccaggagtcagctgtactagcaactgctccaaggatagatgatgaaatcccccctccacttcctgtacggacacctgaatcatttattgtggttgaggaagctggagaattctcaccaaatgttcccaaatccttatcctcagctgtgaaggtaaaaattggaacatcactggaatggggtggaacatctgaaccaaagaaatttgatgactctgtgatacttagaccaagcaagagtgtaaaactccgaagtcctaaatcagaactacatcaagatcgttcttctcccccacctcctctcccagaaagaactctagagtccttctttcttgccgatgaagattgtatgcaggcccaatctatagaaacatattctactagctatcctgacaccatggaaaattcaacatcttcaaaacagacactgaagactcctggaaaaagtttcacaaggagtaagagtttgaaaattttgcgaaacatgaaaaagagtatctgtaattcttgcccaccaaacaagcctgcagaatctgttcagtcaaataactccagctcatttctgaattttggttttgcaaaccgtttttcaaaacccaaaggaccaaggaatccaccaccaacttggaatatttaataaaactcagatttataataatatgggctgcaagtacacctgcaaataaaactactagaatactgctagttaaaataagtgctctatatgcataatatgaagatatgctaatgtgttaatagcttttaaaagaaaagcaaaatgccaataagtgccagttttgcattttcatatcatttgcattgagttgaaaactgcaaataaaagtttgtcacttgagcttatgtacagaatgctatatgagaaacacttttagaatggatttatttttcatttttgccagttatttttattttcttttacttttctacataaacataaacttcaaaaggtttgtaagatttggatctcaactaatttctacattgccagaatatactataaaaagttaaaaaaaaaaacttactttgtgggttgcaatacaaactgctcttgacaatgactattccctgacagttatttttgcctaaatggagtataccttgtaaatcttcccaaatgttgtggaaaactggaatattaagaaaatgagaaattatatttattagaataaaatgtgcaaataatgacaattatttgaatgtaacaag

SEQ ID NO: 2 - protein, 808 aa, Homo sapiens
MDQREILQKFLDEAQSKKITKEEFANEFLKLKRQSTKYKADKTYPTTVAENAKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIVDYTWMLLKDGIIPENFSVFSLIREMRTQRPSLVQTQEQYELVYNAVLELFKRQMDVIRDKHSGTESQAKHCIPEKNHTLQADSYSPNLPKSTTKAAKMMNQQRTKMEIKESSSFDFRTSEISAKEELVLHPAKSSTSFDFLELNYSFDKNADTTMKWQTKAFPIVGEPLQKHQSLDLGSLLFEGCSNSKPVNAAGRYFNSKVPITRTKSTPFELIQQRETKEVDSKENFSYLESQPHDSCFVEMQAQKVMHVSSAELNYSLPYDSKHQIRNASNVKHHDSSALGVYSYIPLVENPYFSSWPPSGTSSKMSLDLPEKQDGTVFPSSLLPTSSTSLFSYYNSHSSLSLNSPTNISSLLNQESAVLATAPRIDDEIPPPLPVRTPESFIVVEEAGEFSPNVPKSLSSAVKVKIGTSLEWGGTSEPKKFDDSVILRPSKSVKLRSPKSELHQDRSSPPPPLPERTLESFFLADEDCMQAQSIETYSTSYPDTMENSTSSKQTLKTPGKSFTRSKSLKILRNMKKSICNSCPPNKPAESVQSNNSSSFLNFGFANRFSKPKGPRNPPPTWNI

SEQ ID NO: 3 - DNA, 2356 bp, Homo sapiens
  CDS (42)..(2117)
tccctcaacctacttatagactatttttcttgctctgcagcatggaccaaagagaaattctgcagaagttcctggatgaggcccaaagcaagaaaattactaaagaggagtttgccaatgaatttctgaagctgaaaaggcaatctaccaagtacaaggcagacaaaacctatcctacaactgtggctgagaatgccaagaatatcaagaaaaacagatataaggatattttgccctatgattatagccgggtagaactatccctgataacctctgatgaggattccagctacatcaatgccaacttcattaagggagtttatggacccaaggcttatattgccacccagggtcctttatctacaaccctcctggacttctggaggatgatttgggaatatagtgtccttatcattgttatggcatgcatggagtatgaaatgggaaagaaaaagtgtgagcgctactgggctgagccaggagagatgcagctggaatttggccctttctctgtatcctgtgaagctgaaaaaaggaaatctgattatataatcaggactctaaaagttaagttcaatagtgaaactcgaactatctaccagtttcattacaagaattggccagaccatgatgtaccttcatctatagaccctattcttgagctcatctgggatgtacgttgttaccaagaggatgacagtgttcccatatgcattcactgcagtgctggctgtggaaggactggtgttatttgtgctattgttgattatacatggatgttgctaaaagatgggataattcctgagaacttcagtgttttcagtttgatccgggaaatgcggacacagaggccttcattagttcaaacgcaggaacaatatgaactggtctacaatgctgtattagaactatttaagagacagatggatgttatcagagataaacattctggaacagagagtcaagcaaagcattgtattcctgagaaaaatcacactctccaagcagactcttattctcctaatttaccaaaaagtaccacaaaagcagcaaaaatgatgaaccaacaaaggacaaaaatggaaatcaaagaatcttcttcctttgactttaggacttctgaaataagtgcaaaagaagagctagttttgcaccctgctaaatcaagcacttcttttgactttctggagctaaattacagttttgacaaaaatgctgacacaaccatgaaatggcagacaaaggcatttccaatagttggggagcctcttcagaagcatcaaagtttggatttgggctctcttttgtttgagggatgttctaattctaaacctgtaaatgcagcaggaagatattttaattcaaaggtgccaataacacggaccaaatcaactccttttgaattgatacagcagagagaaaccaaggaggtggacagcaaggaaaacttttcttatttggaatctcaaccacatgattcttgttttgtagagatgcaggctcaaaaagtaatgcatgtttcttcagcagaactgaattattcactgccatatgactctaaacaccaaatacgtaatgcctctaatgtaaagcaccatgactctagtgctcttggtgtatattcttacatacctttagtggaaaatccttatttttcatcatggcctccaagtggtaccagttctaagatgtctcttgatttacctgagaagcaagatggaactgtttttccttcttctctgttgccaacatcctctacatccctcttctcttattacaattcacatagttctttatcactgaattctccaaccaatatttcctcactattgaaccaggagtcagctgtactagcaactgctccaaggatagatgatgaaatcccccctccacttcctgtacggacacctgaatcatttattgtggttgaggaagctggagaattctcaccaaatgttcccaaatccttatcctcagctgtgaaggtaaaaattggaacatcactggaatggggtggaacatctgaaccaaagaaatttgatgactctgtgatacttagaccaagcaagagtgtaaaactccgaagtcctaaatcaggtaaaaatttctcttggctttagatgacatttagccctaagattggaagaatggttcgttaagtttagagtaattcacttcaggaagttacttggttcccataatagcttccagtattcattgatttatttctggctttcccagactagaaattttgtaaagagtcatgggggaagctagggctaaccagaaaataaaataaaaataatgggataaaaaatcggaactactgttttccccctagtcggagcacatccgg

SEQ ID NO: 4 - protein, 692 aa, Homo sapiens
MDQREILQKFLDEAQSKKITKEEFANEFLKLKRQSTKYKADKTYPTTVAENAKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIVDYTWMLLKDGIIPENFSVFSLIREMRTQRPSLVQTQEQYELVYNAVLELFKRQMDVIRDKHSGTESQAKHCIPEKNHTLQADSYSPNLPKSTTKAAKMMNQQRTKMEIKESSSFDFRTSEISAKEELVLHPAKSSTSFDFLELNYSFDKNADTTMKWQTKAFPIVGEPLQKHQSLDLGSLLFEGCSNSKPVNAAGRYFNSKVPITRTKSTPFELIQQRETKEVDSKENFSYLESQPHDSCFVEMQAQKVMHVSSAELNYSLPYDSKHQIRNASNVKHHDSSALGVYSYIPLVENPYFSSWPPSGTSSKMSLDLPEKQDGTVFPSSLLPTSSTSLFSYYNSHSSLSLNSPTNISSLLNQESAVLATAPRIDDEIPPPLPVRTPESFIVVEEAGEFSPNVPKSLSSAVKVKIGTSLEWGGTSEPKKFDDSVILRPSKSVKLRSPKSGKNFSWL

SEQ ID NO: 5 - protein, 802 aa, Mus musculus
MDQREILQQLLKEAQKKKLNSEEFASEFLKLKRQSTKYKADKIYPTTVAQRPKNIKKNRYKDILPYDHSLVELSLLTSDEDSSYINASFIKGVYGPKAYIATQGPLSTTLLDFWRMIWEYRILVIVMACMEFEMGKKKCERYWAEPGETQLQFGPFSISCEAEKKKSDYKIRTLKAKFNNETRIIYQFHYKNWPDHDVPSSIDPILQLIWDMRCYQEDDCVPICIHCSAGCGRTGVICAVDYTWMLLKDGIIPKNFSVFNLIQEMRTQRPSLVQTQEQYELVYSAVLELFKRHMDVISDNHLGREIQAQCSIPEQSLTVEADSCPLDLPKNAMRDVKTTNQHSKQGAEAESTGGSSLGLRTSTMNAEEELVLHSAKSSPSFNCLELNCGCNNKAVITRNGQARASPVVGEPLQKYQSLDFGSMLFGSCPSALPINTADRYHNSKGPVKRTKSTPFELIQQRKTNDLAVGDGFSCLESQLHEHYSLRELQVQRVAHVSSEELNYSLPGACDASCVPRHSPGALRVHLYTSLAEDPYFSSSPPNSADSKMSFDLPEKQDGATSPGALLPASSTTSFFYSNPHDSLVMNTLTSFSPPLNQETAVEAPSRRTDDEIPPPLPERTPESFIVVEEAGEPSPRVTESLPLVVTFGASPECSGTSEMKSHDSVGFTPSKNVKLRSPKSDRHQDGSPPPPLPERTLESFFLADEDCIQAQAVQTSSTSYPETTENSTSSKQTLRTPGKSFTRSKSLKIFRNMKKSVCNSSSPSKPTERVQPKNSSSFLNFGFGNRFSKPKGPRNPPSAWNM

SEQ ID NO: 6 - DNA, 82 bp, Homo sapiens
aaactccgaagtcctaaatcaggtaaaaatttctcttggctttagatgacatttagccctaagattggaagaatggttcgtt

SEQ ID NO: 7 - protein, 14 aa, Homo sapiens
KLRSPKSGKNFSWL